(19) United States
(12) Patent Application Publication
(10) Pub. No.: US 2022/0386905 A1
(43) Pub. Date: Dec. 8, 2022

(54) SIGNAL PROCESSING ALGORITHM FOR IMPROVING ACCURACY OF A CONTINUOUS GLUCOSE SENSOR AND A COMBINED CONTINUOUS GLUCOSE SENSOR AND INSULIN DELIVERY CANNULA

(71) Applicant: Pacific Diabetes Technologies Inc, Portland, OR (US)

(72) Inventors: Peter G. Jacobs, Portland, OR (US); Clara Mosquera-Lopez, Portland, OR (US); Nichole Tyler, Portland, OR (US); Robert S. Cargill, Portland, OR (US); Thomas L. Seidl, Wilsonville, OR (US); William Kenneth Ward, Portland, OR (US)

(21) Appl. No.: 17/684,015

(22) Filed: Mar. 1, 2022

Related U.S. Application Data
(60) Provisional application No. 63/155,726, filed on Mar. 2, 2021.

(51) Int. Cl. A61B 5/145; A61M 5/172; G16H 40/63

(52) U.S. Cl. CPC A61B 5/14532; A61M 5/1723; G16H 40/63; G16H 20/17

(57) ABSTRACT

The present disclosure provides methods of measuring of an analyte in a subject to remove a measurement artifact by using a forecasting model to determine the true analyst concentration in a subject. Also herein, the present disclosure provides parameters and models to estimate the true analyte concentration in a subject.

### CROSS-REFERENCE

This application claims the benefit of U.S. Provisional Application No. 63/155,726 filed Mar. 2, 2021, which is incorporated by reference herein in its entirety.

### STATEMENT AS TO FEDERALLY SPONSORED RESEARCH

This invention was made with government support under Contract number DK101044 awarded by National Institutes of Health. The government has certain rights in the invention.

### SUMMARY OF THE INVENTION

Provided herein, one embodiment of the disclosure provides a method for estimating a true analyte concentration in a subcutaneous space, comprising (a) measuring a first concentration of an analyte in interstitial fluid in the subcutaneous space using a sensor at given time; (b) predicting a second concentration of the analyte in the interstitial fluid at the given time using a forecasting model; and (c) combining the first concentration of the analyte and the second concentration of the analyte in a signal processing module to estimate a true analyte concentration, wherein steps (a)-(c) are repeated at least one time during a period of time following delivery of a composition near the sensor, wherein steps (a)-(c) are repeated to mitigate a dilution artifact caused by the delivery of the composition near the sensor.

In some embodiments, the sensor is located near a subcutaneous delivery site of a composition. In some embodiments, the period of time is at least about 25 minutes. In some embodiments, the period of time is no more than about 45 minutes.

In some embodiments, the delivery of the composition is within about 15 mm of the sensor. In some embodiments, the delivery of the composition is within about 10 mm of the sensor. In some embodiments, the delivery of the composition is within about 7 mm of the sensor.

In some embodiments, the dilution artifact comprises an artifact in a measurement of the first analyte concentration by the sensor due to the delivery of the composition near the sensor. In some embodiments, the delivery of the composition near the sensor results in dilution of the interstitial fluid near the sensor.

In some embodiments, the forecasting model is based at least in part on the first concentration of the analyte, previous analyte concentrations measured by the sensor, delivery of the composition, or any combination thereof. In some embodiments, delivery of the composition near the sensor is identified by a notification from a device configured to deliver the composition, wherein the device is communicably coupled to the forecasting model. In some embodiments, the signal processing module combines the first concentration of the analyte and the second concentration of the analyte as a weighted sum to estimate a true analyte concentration.

In some embodiments, the weighted sum is based at least in part on covariance of the first concentration of the analyte and the second concentration of the analyte.

In some embodiments, the composition is delivered by a tubed pump or a patch pump using a delivery system. In some embodiments, the delivery system comprises a continuous infusion pump.

In some embodiments, the delivery system comprises an open loop delivery system. In some embodiments, the delivery system comprises a closed loop delivery system. In some embodiments, the delivery system comprises a hybrid closed loop delivery system. In some embodiments, the composition is delivered by subcutaneous injection.

In some embodiments, the forecasting model is configured to compensate for the dilution artifact during delivery of the composition. In some embodiments, the second concentration of the analyte is provided by the forecasting model in real-time to mitigate the dilution artifact.

In some embodiments, the forecasting model comprises a machine learning model, an ordinary differential equation (ODE)-based model, or a combination thereof. In some embodiments, the machine learning model comprises a linear regression model, support vector regression model, multivariable adaptive regressive spline model, neural network model, ridge regression model, Lasso regression model, ElasticNet regression model. In some embodiments, the neural network model comprises a convolutional neural network layer, a recurrent neural network layer, or a combination thereof. In some embodiments, the recurrent neural network layer comprises a long-short term memory. In some embodiments, the ODE-based model comprises an ODE solver to solve a system of ODEs for a time of interest. In some embodiments, the system of ODEs comprises kinetics and/or dynamics of the analyte, the composition, or a combination thereof. In some embodiments, the kinetics and/or dynamics comprise kinetics and/or dynamics of insulin, glucagon, a carbohydrate, pramlintide, glucose, or any combination thereof. In some embodiments, the ODE-based model comprises an metabolism regulatory model. In some embodiments, the metabolism regulatory model comprises a glucoregulatory model. In some embodiments, the forecasting model is obtained by training a machine learning model. In some embodiments, the machine learning model comprises a neural network. In some embodiments, training the machine learning model comprises using training data comprising analyte sensor measurements from a population with a disease or disorder.

In some embodiments, the disease or disorder comprises an insulin resistance, Type 1 diabetes mellitus, or Type 2 diabetes mellitus.

In some embodiments, the training data comprises the analyte sensor measurements for about 3 hours prior to a time of interest. In some embodiments, the method further comprises processing the first concentration of the analyte using a filter to remove noise after (a). In some embodiments, the filter comprises a low-pass filter, a bandpass filter, a high pass filter, or a combination thereof.

In some embodiments, the first concentration of the analyte and the second concentration of the analyte are combined in (c) using a filter. In some embodiments, the filter comprises a Kalman filter, extended Kalman filter, or sigma point Kalman filter. In some embodiments, the first concentration of the analyte is weighted based at least in part on the variance of the measurement.

In some embodiments, the first concentration of the analyte is weighted more heavily if the variance of the first concentration of the analyte is low. In some embodiments, the second concentration of the analyte is weighted more heavily if the variance of the first concentration of the analyte is high. n some embodiments, further comprises providing the true analyte concentration to a user. In some embodiments, the true analyte concentration comprises the first analyte concentration, the second analyte concentration, or a combination thereof.

In some embodiments, the user has a disease or disorder comprising insulin resistance. In some embodiments, the disease or disorder comprises Type 1 diabetes mellitus. In some embodiments, the disease or disorder comprises Type 2 diabetes mellitus.

In some embodiments, the sensor is implanted in the subcutaneous space. In some embodiments, the sensor comprises a continuous amperometric glucose sensor.

In some embodiments, the analyte comprises a carbohydrate. In some embodiments, the carbohydrate comprises glucose. In some embodiments, the composition comprises a hormone. In some embodiments, delivery of a composition comprises delivery of an insulin bolus. In some embodiments, the hormone comprises insulin, glucagon, pramlintide, or any combination thereof.

In some embodiments, the composition further comprises at least one pharmaceutical acceptable excipient. In some embodiments, the at least one pharmaceutical acceptable excipient comprises phenol, cresol, a salts, a stabilizing agent, or any combination thereof.

Provided herein, one embodiment of the disclosure provides a method for estimating a true analyte concentration in a subcutaneous space, comprising (a) measuring a first concentration of an analyte in interstitial fluid in the subcutaneous space using a sensor at given time; (b) predicting a second concentration of the analyte in the interstitial fluid at the given time using a forecasting model, wherein the forecasting model comprises a machine learning model and a metabolism regulatory model; and (c) combining the first concentration of the analyte and the second concentration of the analyte in a signal processing module to estimate a true analyte concentration.

In some embodiments, the metabolism regulatory model comprises a glucoregulatory model.

In some embodiments, the machine learning comprises a comprises a linear regression model, support vector regression model, multivariable adaptive regressive spline model, neural network model, ridge regression model, Lasso regression model, ElasticNet regression model.

In some embodiments, the neural network model comprises a convolutional neural network layer, a recurrent neural network layer, or a combination thereof.

In some embodiments, the recurrent neural network layer comprises an long-short term memory. In some embodiments, the method further comprises processing the first concentration of the analyte using a filter to remove noise after (a). In some embodiments, the filter comprises a low-pass filter, a bandpass filter, a high pass filter, or a combination thereof. In some embodiments, the first concentration of the analyte and the second concentration of the analyte are combined in (c) using a filter. In some embodiments, the filter comprises a Kalman filter, extended Kalman filter, or sigma point Kalman filter. In some embodiments, the first concentration of the analyte is weighted based at least in part on the variance of the measurement. In some embodiments, the first concentration of the analyte is weighted more heavily if the variance of the first concentration of the analyte is low. In some embodiments, the second concentration of the analyte is weighted more heavily if the variance of the first concentration of the analyte is high. In some embodiments, further comprises providing the true analyte concentration to a user. In some embodiments, the true analyte concentration comprises the first analyte concentration, the second analyte concentration, or a combination thereof.

In some embodiments, the user has a disease or disorder comprising insulin resistance. In some embodiments, the disease or disorder comprises Type 1 diabetes mellitus. In some embodiments, the disease or disorder comprises Type 2 diabetes mellitus.

In some embodiments, the sensor is implanted in the subcutaneous space. In some embodiments, the sensor comprises a continuous amperometric glucose sensor.

In some embodiments, the analyte comprises a carbohydrate. In some embodiments, the carbohydrate comprises glucose. Provided herein, one embodiment of the disclosure provides a system for estimating a true analyte concentration in a subcutaneous space, comprising: (a) a sensor for measuring a first concentration of an analyte in interstitial fluid in the subcutaneous space; (b) a forecasting model configured to predict a second concentration of the analyte in the interstitial fluid; and (c) a signal processing module configured to combine the first concentration of the analyte and the second concentration of the analyte to estimate a true analyte concentration, wherein the true analyte concentration is estimated during a period of time following delivery of a composition near of the sensor, wherein the true analyte concentration is estimated to mitigate a dilution artifact caused by the delivery of the composition near the sensor.

Provided herein, one embodiment of the disclosure provides a system for estimating a true analyte concentration in a subcutaneous space, comprising: (a) a sensor for measuring a first concentration of an analyte in interstitial fluid in the subcutaneous space; (b) a forecasting model configured to predict a second concentration of the analyte in the interstitial fluid, wherein the forecasting model comprises a machine learning model and a metabolism regulatory model; and (c) a signal processing module configured to combine the first concentration of the analyte and the second concentration of the analyte to estimate a true analyte concentration.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1B demonstrate the calibration results from aa participant from a clinical study saline proximal sensor. Linear regression smooths raw sensor measurements, while Kalman filtering shifts the signal to account for plasma-interstitium delay (top). Plot of glucose sensor for a subject after single-point calibration with Kalman filter adjustments (bottom).

FIG. 2: This graph summarizes the results of a study of 7 persons with type 1 diabetes carried out at Oregon Health and Science University (OHSU).

FIG. 3: The upper panel shows a typical example of dilution artifact in a person with type 1 diabetes. Measured glucose data obtained from a subcutaneous CGM is shown in the solid line and shows a rapid decline in the glucose data immediately after a bolus of insulin, which is given at time 0. The measurement was taken from the situation in which a substantially-sized insulin bolus is given. The magnitude and duration of the error is directly proportional to the size of the bolus. A small bolus such as 4-7 units or less generally has an error that lasts less than 15 minutes. The forecasted glucose (which is predicted using either a glucoregulatory model or a machine learning model along with a Kalman filter), shown as a dashed line, is the glucose data obtained by a glucoregulatory model, and is the data shown to the patient instead of measured glucose stream during the period of dilution artifact. As shown in the lower panel, this metric is continuously evaluated by the algorithm. In this example, the algorithm determines that when the metric falls to 12% (after initially rising), the displayed glucose value reverts to the measured value. Setting the metric to 12% illustrates one example; this decision metric can also be set to other levels. The 12% criterion is shown as a black horizontal line.

FIG. 4: The upper panel shows an atypical example of dilution artifact in a person with type 1 diabetes. In this example, there is a rise in the measured glucose value, not the more typical decline. Measured glucose data obtained from a subcutaneous CGM is shown in the solid line and shows a rapid increase in the glucose data immediately after a bolus of insulin, which is given at time 0. The forecasted glucose, shown as a dashed line, as before, is the glucose data obtained by a glucoregulatory model. The lower panel plots the metric, |(M-F)|/F, defined above. Once again, the algorithm determines that when the metric falls to 12% (after initially rising), the displayed glucose value reverts to the measured. This 12% criterion is only one example: it can be set to other levels. It should also be mentioned that one can convert glucose concentration (in mM or mmol/liter) to mg/dl by multiplying by 18.

FIG. 5: Survival curve showing the percentage of observations after fluid delivery (insulin or PBS solution) that are free of the dilution artifact vs. time since the fluid was delivered.

FIG. 6 shows an embodiment of a combination sensor and insulin delivery cannula in which there are two indicating electrodes, as labeled.

FIGS. 7A-7B: The Jacobs laboratory at OHSU has developed a glucoregulatory model, as described in detail in Example 1. Two examples of actual reference blood glucose data are shown as dashed lines, referred to as “Plant”. The model-predicted glucose levels are shown as solid lines, referred to as “Model”.

FIG. 8: An example architecture of an LSTM model used for forecasting glucose 30 and 60 minutes in the future. This LSTM model was used to demonstrate that use of a model like this could be used to mitigate the dilution artifact caused by insulin delivery near a glucose sensing location.

FIG. 9 shows an LSTM neural network to predict glucose 30 minutes in the future in people with type 1 diabetes using a closed-loop control algorithm.

FIG. 10 shows a general schematic of a computer system for use in the present disclosure.

### DETAILED DESCRIPTION OF THE INVENTION

The present disclosure provides signal processing methods for improving the accuracy of a continuous glucose monitor (CGM) including the situation in which the CGM is designed to be integrated in close proximity with a cannula that is delivering insulin. Glucose values measured by an amperometric sensor can be noisy and thus is filtered. This disclosure describes filtering methods for improving the accuracy of these sensor measurements. Noise can also be introduced on a glucose sensor if formulations of insulin (or other drugs) are delivered within close proximity to the site of glucose measurement. A glucose forecasting model can be used to improve the accuracy of the glucose measurement following delivery of insulin and other hormones. The methods of the present disclosure describe how to use filtering methodologies in combination with a glucose forecasting model that predict future glucose values to improve the accuracy of the glucose measurement. The model forecast of glucose can then be optimally combined with an observed or measured CGM value using a Kalman filter to provide a final estimate of glucose.

With the proper choice of sensing chemistry layers, it is possible to operate a continuous glucose monitor (CGM) in the immediate vicinity of the delivery of subcutaneous insulin. The glucose monitoring error in the vicinity of the insulin formulation is no greater than error in the vicinity of the same volume of a control saline solutions. However, when any liquid is delivered in the interstitial fluid in presence of a CGM, there is the potential for artifact due to the dilution. Though this dilution artifact is not lengthy (the fluid is absorbed quickly, resolving the dilution artifact), it should not be ignored. The direction of the dilution artifact can be negative, resulting in an underestimate of the true glucose level. There is a need for a method to appropriately compensate for interstitial dilution. The disclosure provides several methods for such compensation, such as filtering methodologies and also methods whereby model-based forecasted glucose values are utilized during a period of time following an insulin bolus. The glucose values displayed to a user of the sensing cannula are determined by both the measured glucose and the forecasted glucose values during periods of expected dilution artifacts present in the CGM signal. The forecasting model may be a machine learning model or a glucoregulatory model comprised of ordinary differential equations (ODEs).

### Definitions

Unless otherwise defined herein, scientific and technical terms used in connection with the present disclosure shall have the meanings that are commonly understood by those of ordinary skill in the art. The meaning and scope of the terms should be clear, however, in the event of any latent ambiguity, definitions provided herein take precedent over any dictionary or extrinsic definition. Further, unless otherwise required by context, singular terms shall include pluralities and plural terms shall include the singular. In this application, the use of “or” means “and/or” unless stated otherwise. Furthermore, the use of the term “including”, as well as other forms, such as “includes” and “included”, is not limiting.

Reference throughout this specification to “some embodiments,” “further embodiments,” or “a particular embodiment,” means that a particular feature, structure, or characteristic described in connection with the embodiment is included in at least one embodiment. Thus, the appearances of the phrase “in some embodiments,” or “in further embodiments,” or “in a particular embodiment” in various places throughout this specification are not necessarily all referring to the same embodiment. Furthermore, the particular features, structures, or characteristics may be combined in any suitable manner in one or more embodiments.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

That the present disclosure may be more readily understood, select terms are defined below.

By the term, “settling time” it is meant as the time after an insulin bolus that it takes for the measured glucose values measured by a continuous glucose monitor (CGM), which initially are affected by dilution artifact, to return to the true glucose value without such artifact.

By the term, “measured CGM Values,” it is meant, values that are the glucose estimates provided by the CGM in the presence of dilution artifact caused by insulin delivery near a glucose sensing location. There is a degree of inaccuracy in measured CGM values until the fluid causing the dilution has migrated away from the sensor and into the surrounding tissue.

By the term, “interstitial dilution compensation,” it is meant a technique whereby the CGM glucose values obtained in real time are not shown to the patient. Instead, calculations are carried out that lead to generation of forecasted glucose values which are shown to the patient.

By the term, “Forecasted Glucose Values,” it is meant the calculated values that are shown to the patient (user) for a period of time in combination with an actual CGM value or instead of showing the actual CGM values. The forecasted glucose values minimize dilution artifact and therefore are more accurate than the CGM values until setting has completed. Forecasted glucose values can be calculated in several ways, as taught below, and are closer to the true systemic interstitial glucose values than the values that are obtained locally from the CGM in the presence of dilution artifact.

By the term, “Glucoregulatory Model,” it is meant a mathematical model comprised of differential equations whereby compartments of the physical system (e.g. amount of glucose in plasma) are represented by state equations. The glucoregulatory model can be used to calculate a forecasted glucose value using input features including previous CGM and insulin dosed to the patient. At the very least, such a model accounts for movement of glucose from the gut and/or liver into the bloodstream (such as the rate of appearance) and from the bloodstream into mammalian cells and tissues (such as, glucose disposal, glucose uptake, or rate of disappearance). The rate at which the liver and kidney discharge glucose into the bloodstream is known as glucose production rate. Some glucoregulatory models also separate glucose uptake into insulin-mediated glucose uptake (for example, when glucose is taken up by fat and muscle cells) and non-insulin-mediated glucose uptake (for example, when glucose is taken up into the brain).

By the term, “Combination Insulin Delivery-Glucose Sensing Device (CIDGS device),” it is meant an integrated insulin delivery and CGM glucose sensing device in which the indicating electrode of the CGM is located near the point of insulin delivery.

By the term, “Machine Learning Model,” is it meant a data-driven black-box model that is trained on a training data set to calculate forecasted glucose measurements. Examples of the types of machine learning models that are referred to in this disclosure include, but are limited to, a neural network, linear regression, support vector regression, and multivariable adaptive regressive spline (MARS) models. A machine learning model can be used to calculate forecasted glucose values when a dilution artifact may be contributing to substantial error in the sensor measurement following an insulin bolus delivery.

By the term, “Kalman Filter,” it is meant a method that can be used to balance prediction estimates from a model with measured observation data. The Kalman filter estimates an outcome using both the model estimate and the noisy observation, and depending on the noise in the model and the noise in the measurement, determine how best to estimate the outcome using a noise-dependent weighting factor called the Kalman gain. In this work, the Kalman filter is used to predict glucose using both a noisy sensor measurement made after an insulin dose and a model estimation of the glucose.

By the term, “Linear regression model” it is meant a filter that can be used to predict a future glucose value using prior glucose values. A regression model can be fit to a set of measured glucose data over a window of time (e.g., 5 minutes) to determine a slope and intercept of the data. This regression model may then be used to forecast glucose in the future.

By the term, “open loop delivery system,” it is meant a system in which the user determines the amount of insulin to inject, as well as, determination of the approximate amount of food to be consumed.

By the term, “closed loop delivery system,” it is meant a system in which no used input is required. For instance, such a system would monitor the insulin requirement in real-time and administer the appropriate insulin dosage insulin pump in which no user input is required in response to reading from a display. In some instances, the system uses an external device to automatically adjust the insulin delivery of the pump based on the glucose readings from a continuous glucose sensor.

By the term, “hybrid delivery system,” it is meant a system in which user input and computer controlled calculations are integrated into a single system. In such instances, an algorithm is designed to calculate automated insulin delivery alongside manual mealtime boluses. In some instances, this may be any insulin pump able to deliver variable (automated) basal insulin by using an algorithm and real-time CGM sensor glucose trends. The system continuously monitors blood glucose levels and calculates the amount of insulin required. Then, it automatically adjusts the background, or basal, insulin based on your blood sugar readings.

Continuous subcutaneous insulin infusion (SCII) is a process by which fast-acting insulin is delivered under the skin to a person with insulin-treated Type 1 or Type 2 diabetes mellitus. The delivery can be truly continuous or can be delivered on a nearly-continuous basis by delivery of a series of frequent microboluses. For example, if a pump is programmed to deliver a basal rate of 1 unit per hour, and each microbolus is 0.05 unit, then the pump will deliver a microbolus 20 times per hour, e.g., every 3 minutes. Even though such delivery is not fully continuous, it is nonetheless included in the broad category of “continuous subcutaneous insulin infusion.”

Some insulin pumps are known as tubed pumps. In such cases, the actual pump unit (which includes the insulin reservoir) can be carried in a pocket, on the belt, suspended from a lanyard around the neck, or located elsewhere. In such cases, a tube (usually made from a polymer), through which insulin flows, serves as the fluid connection between the pump unit and the cannula (metal or polymer) inserted under the skin.

Insulin pumps without tubes are known as patch pumps, tubeless pumps, or on-the-body pumps. In such cases, the pump is affixed to the skin of the body, typically with the aid of an adhesive. Because there is no need to carry the pump unit in a pocket or on a belt, on-the-body devices are often considered to be more convenient for athletic patients who enjoy activities, such as running, sports, yoga, or gym activities.

The on-the-body device contains a chamber (reservoir) that contains sufficient insulin for several days. This reservoir can be prefilled at the time of purchase or can be filled by the user. The motive force that forces the insulin into the cannula can be a motor, a spring, a change in osmotic pressure, or a contractile (“muscle”) wire made from nitinol, which changes length when an electrical current is applied. Other types of motive force mechanisms also exist.

The cannula in an on-the-body pump can be inserted by one of several methods. For example, the pump can be first be adhered to the skin of the body, then a spring or motor can be activated to insert the cannula into the skin, in some cases accompanied by an internal retractable stylet or a retractable external trocar. In other cases, the cannula and the housing of the pump are first adhered to the skin without the actual pump, which is attached and inserted at a later time. In other words, there are two types of on-the-body pumps, one that is a single unified device and one whose cannula and housing are separate from, but attachable to, the pump unit that contains the insulin reservoir.

In some cases, the operation of the pump is controlled by a separate hand-held unit that communicates with the pump unit by telemetry. Using this hand-held device, the user can manually deliver a bolus of insulin or can maintain or change the basal rate of insulin delivery. This hand-held device can contain software formulas that have been entered for calculation of meal-related boluses (insulin:carbohydrate ratio), hyperglycemia correction boluses (how much insulin is needed based on the magnitude of hyperglycemia), and basal insulin rates for different times of day. In addition, the hand-held unit will typically keep track of insulin-on-board, which is an estimate of the amount of administered insulin that is still in the body, but has not yet exerted its effect on peripheral insulin receptors. The knowledge of insulin-on-board is very useful to the patient with diabetes because it discourages him or her from overdelivering insulin before earlier doses have had the time to exert their full effect. This phenomenon of overdelivering before completion of earlier dose effects is known as insulin stacking.

In some cases, a small pump is adhered to the skin of the body but is not directly connected to a subcutaneous cannula. Instead, such a device can be connected to a short tube that is itself connected to the subcutaneous cannula. Such a device is an on-the-body pump even though it has a short tube.

### Method of Estimating True Analyte Concentration

The method of the present disclosure can be used to mitigate the observation of an artifact. In some instances, the artifact is due to dilution (i.e., a dilution artifact). In some embodiments, the dilution artifact is an artifact in a measurement of a first analyte concentration by the senor due to the delivery of the composition near the sensor.

Provided herein is a method for estimating a true analyte concentration in the subcutaneous space, comprising: measuring a first concentration of an analyte in interstitial fluid in the subcutaneous space using a sensor at given time; predicting a second concentration of the analyte in the interstitial fluid at the given time using a forecasting model; and combining the first concentration of the analyte and the second concentration of the analyte in a signal processing module as a weighted sum to estimate a true analyte concentration. In some instances, the weighted sum is based at least in part on covariance of the first concentration of the analyte and the second concentration of the analyte. In some instances, the steps are repeated at least one time during a period of time following delivery of a composition near the sensor.

The period of time following delivery of a composition to measure the true analyte concentration following the delivery of a composition can be varied in order to provide an accurate measurement. In some embodiments, the period of time is about 1 minute to about 60 minutes. In some embodiments, the period of time is about 1 minute to about 5 minutes, about 1 minute to about 10 minutes, about 1 minute to about 15 minutes, about 1 minute to about 20 minutes, about 1 minute to about 25 minutes, about 1 minute to about 30 minutes, about 1 minute to about 35 minutes, about 1 minute to about 40 minutes, about 1 minute to about 45 minutes, about 1 minute to about 50 minutes, about 1 minute to about 55 minutes, about 1 minute to about 60 minutes, about 5 minutes to about 10 minutes, about 5 minutes to about 15 minutes, about 5 minutes to about 20 minutes, about 5 minutes to about 25 minutes, about 5 minutes to about 30 minutes, about 5 minutes to about 35 minutes, about 5 minutes to about 40 minutes, about 5 minutes to about 45 minutes, about 5 minutes to about 50 minutes, about 5 minutes to about 55 minutes, about 5 minutes to about 60 minutes, about 10 minutes to about 15 minutes, about 10 minutes to about 20 minutes, about 10 minutes to about 25 minutes, about 10 minutes to about 30 minutes, about 10 minutes to about 35 minutes, about 10 minutes to about 40 minutes, about 10 minutes to about 45 minutes, about 10 minutes to about 50 minutes, about 10 minutes to about 55 minutes, about 10 minutes to about 60 minutes, about 15 minutes to about 20 minutes, about 15 minutes to about 25 minutes, about 15 minutes to about 30 minutes, about 15 minutes to about 35 minutes, about 15 minutes to about 40 minutes, about 15 minutes to about 45 minutes, about 15 minutes to about 50 minutes, about 15 minutes to about 55 minutes, about 15 minutes to about 60 minutes, about 20 minutes to about 25 minutes, about 20 minutes to about 30 minutes, about 20 minutes to about 35 minutes, about 20 minutes to about 40 minutes, about 20 minutes to about 45 minutes, about 20 minutes to about 50 minutes, about 20 minutes to about 55 minutes, about 20 minutes to about 60 minutes, about 25 minutes to about 30 minutes, about 25 minutes to about 35 minutes, about 25 minutes to about 40 minutes, about 25 minutes to about 45 minutes, about 25 minutes to about 50 minutes, about 25 minutes to about 55 minutes, about 25 minutes to about 60 minutes, about 30 minutes to about 35 minutes, about 30 minutes to about 40 minutes, about 30 minutes to about 45 minutes, about 30 minutes to about 50 minutes, about 30 minutes to about 55 minutes, about 30 minutes to about 60 minutes, about 35 minutes to about 40 minutes, about 35 minutes to about 45 minutes, about 35 minutes to about 50 minutes, about 35 minutes to about 55 minutes, about 35 minutes to about 60 minutes, about 40 minutes to about 45 minutes, about 40 minutes to about 50 minutes, about 40 minutes to about 55 minutes, about 45 minutes to about 50 minutes, about 45 minutes to about 55 minutes, about 45 minutes to about 60 minutes, about 50 minutes to about 55 minutes, about 50 minutes to about 60 minutes, or about 55 minutes to about 60 minutes. In some embodiments, the period of time is about 1 minute, about 5 minutes, about 10 minutes, about 15 minutes, about 20 minutes, about 25 minutes, about 30 minutes, about 35 minutes, about 40 minutes, about 45 minutes, about 50 minutes, about 55 minutes, or about 60 minutes. In some embodiments, the period of time is at least about 1 minute, about 5 minutes, about 10 minutes, about 15 minutes, about 20 minutes, about 25 minutes, about 30 minutes, about 35 minutes, about 40 minutes, about 45 minutes, about 50 minutes, about 55 minutes, or about 60 minutes.

The delivery of the composition can occur near the sensor. In some embodiments, the delivery of the composition is within 15 mm, 14 mm, 13 mm, 12 mm, 10 mm, 9 mm, 8 mm, 7 mm, 6 mm, 5 mm of the sensor. In some embodiments, the delivery of the composition is no more than 15 mm from the sensor.

In some embodiments, the method further comprise processing the first concentration of the analyte using a filter, such as those described herein, to remove noise after measuring a first concentration of an analyte in interstitial fluid in the subcutaneous space using a sensor at given time.

### Forecasting Model

The forecasting model described in the present disclosure comprises a model to predict an analyte concentration.

In some embodiments, the forecasting model is based at least in part on the first concentration of the analyte, previous analyte concentrations measured by the sensor, delivery of the composition, or any combination thereof. In some embodiments, the forecasting model is based at least in part on the first concentration of the analyte. In some embodiments, the forecasting model is based at least in part on the previous analyte concentrations measured by the sensor, delivery of the composition. In some embodiments, the forecasting model is based at least in part on the delivery of the composition (e.g., delivery of an insulin bolus near a glucose sensor).

In some embodiments, the forecasting model is configured to compensate for an artifact during delivery of the composition. In some embodiments, the artifact is a dilution artifact.

In some embodiments, the second concentration of the analyte is provided by the forecasting model in real-time to mitigate the dilution artifact.

In some embodiments, the forecasting model comprises a machine learning model. In some cases, the forecasting model comprises a linear regression model, support vector regression model, multivariable adaptive regressive spline model, neural network model, ridge regression model, Lasso regression model, or ElasticNet regression model. In some instances, the neural network model comprises a convolutional neural network layer or a recurrent neural network layer. In some examples, the recurrent neural network layer comprises a long short-term memory (LSTM).

In some embodiments, the forecasting model is obtained by training a machine learning model. In some embodiments, the machine learning model comprises a neural network, such as those described herein. In some embodiments, the training of the machine learning model comprises using training data comprising analyte sensor measurements from a population with a disease or disorder. As an example, the training data can comprise glucose measurements from a population with Type I or Type II diabetes mellitus.

In some embodiments, the training data comprises the analyte sensor measurements for about 3 hours prior to a time of interest. In such an example, a glucose concentration may be predicted using glucose measurements from about 3 hours prior from a population with Type I or Type II diabetes mellitus.

In some embodiments, the forecasting model comprises an ordinary differential equation (ODE)-based model. In some embodiments, the ODE-based model comprises an ODE solver to solve a system of ODEs for a time of interest. In some embodiments, the system of ODEs comprises kinetics and/or dynamics of the analyte, the composition, or a combination thereof. In some embodiments, the kinetics and/or dynamics comprise kinetics and/or dynamics of insulin, glucagon, a carbohydrate, pramlintide, glucose, or any combination thereof.

In some embodiments, the first concentration of the analyte is weighted based at least in part on the variance of the measurement. In some embodiments, the first concentration of the analyte is weighted more heavily if the variance of the first concentration of the analyte is low, such as a value of about 1 nA² to about 50 nA². In some embodiments, a low variance is about 1 nA² to about 50 nA². In some embodiments, a low variance is about 1 nA² to about 5 nA², about 1 nA² to about 10 nA², about 1 nA² to about 15 nA², about 1 nA² to about 20 nA², about 1 nA² to about 30 nA², about 1 nA² to about 40 nA², about 1 nA² to about 50 nA², about 5 nA² to about 10 nA², about 5 nA² to about 15 nA², about 5 nA² to about 20 nA², about 5 nA² to about 30 nA², about 5 nA² to about 40 nA², about 5 nA² to about 50 nA², about 10 nA² to about 15 nA², about 10 nA² to about 20 nA², about 10 nA² to about 30 nA², about 10 nA² to about 40 nA², about 10 nA² to about 50 nA², about 15 nA² to about 20 nA², about 15 nA² to about 30 nA², about 15 nA²to about 40 nA², about 15 nA² to about 50 nA², about 20 nA² to about 30 nA², about 20 nA² to about 40 nA², about 20 nA² to about 50 nA², about 30 nA² to about 40 nA², about 30 nA² to about 50 nA², or about 40 nA² to about 50 nA². In some embodiments, a low variance is about 1 nA², about 5 nA², about 10 nA², about 15 nA², about 20 nA², about 30 nA², about 40 nA², or about 50 nA². In some embodiments, a low variance is at least about 1 nA², about 5 nA², about 10 nA², about 15 nA², about 20 nA², about 30 nA², or about 40 nA². In some embodiments, a low variance is at most about 5 nA², about 10 nA², about 15 nA², about 20 nA², about 30 nA², about 40 nA², or about 50 nA². In some embodiments, the second concentration of the analyte is weighted more heavily if the variance of the first concentration of the analyte is high, such as a value of about 51 nA² to about 100 nA². In some embodiments, a high variance is about 51 nA² to about 100 nA². In some embodiments, a high variance is about 51 nA² to about 60 nA², about 51 nA² to about 70 nA², about 51 nA² to about 80 nA², about 51 nA² to about 85 nA², about 51 nA² to about 90 nA², about 51 nA² to about 95 nA², about 51 nA² to about 100 nA², about 60 nA² to about 70 nA², about 60 nA² to about 80 nA², about 60 nA² to about 85 nA², about 60 nA² to about 90 nA², about 60 nA² to about 95 nA², about 60 nA² to about 100 nA², about 70 nA² to about 80 nA², about 70 nA² to about 85 nA², about 70 nA² to about 90 nA², about 70 nA² to about 95 nA², about 70 nA² to about 100 nA², about 80 nA² to about 85 nA², about 80 nA² to about 90 nA², about 80 nA² to about 95 nA², about 80 nA² to about 100 nA², about 85 nA² to about 90 nA², about 85 nA² to about 95 nA², about 85 nA² to about 100 nA², about 90 nA² to about 95 nA², about 90 nA² to about 100 nA², or about 95 nA² to about 100 nA². In some embodiments, a high variance is about 51 nA², about 60 nA², about 70 nA², about 80 nA², about 85 nA², about 90 nA², about 95 nA², or about 100 nA². In some embodiments, a high variance is at least about 51 nA², about 60 nA², about 70 nA², about 80 nA², about 85 nA², about 90 nA², or about 95 nA². In some embodiments, a high variance is at most about 60 nA², about 70 nA², about 80 nA², about 85 nA², about 90 nA², about 95 nA², or about 100 nA².

### Filter

The method of the present disclosure can use a filter to remove the noise after making a measurement. In some embodiments, the filter comprise a low-pass filter, a bandpass filter, a high pass filter, or a combination thereof.

In some embodiments, the first concentration of the analyte and the second concentration of the analyte are combined using a filter. In some instances, the first concentration of the analyte and the second concentration of the analyte are combined in a signal processing module as a weighted sum to estimate a true analyte concentration.

In some embodiments, the filter comprises a Kalman filter, extended Kalman filter, or sigma point Kalman filter.

In some embodiments, the disease or disorder comprises an insulin resistance, a Type 1 diabetes mellitus, or a Type 2 diabetes mellitus.

In some embodiments, the delivery of the composition near the sensor is identified by a notification from a device configured to deliver the composition. In some instances, the device is communicably coupled to the forecasting model.

In some embodiments, the composition is delivered via a pump. In some embodiments, the composition is delivered by a tubed pump or a patch pump using an open loop delivery system. In some embodiments, the composition is delivered by a tubed pump or a patch pump using a closed loop delivery system. In some embodiments, the composition is delivered by a tubed pump or a patch pump using a hybrid closed loop delivery system.

In some embodiments, the sensor is implanted in the subcutaneous space.

In some embodiments, the analyte is a metabolite, protein, carbohydrate, or biomolecule. In some embodiments, the analyte comprises glucose. In some embodiments, the composition comprises a hormone.

In some embodiments, delivery of a composition comprises delivery of an insulin bolus. In some embodiments, the device comprises a hormone. In some cases, the hormone comprises insulin, glucagon, pramlintide, or any combination thereof. In some embodiments, the composition further comprises at least one pharmaceutical acceptable excipient. In some embodiments, the at least one pharmaceutical acceptable excipient comprises phenol, cresol, a salts, a stabilizing agent, or any combination thereof.

Multi-Chamber Pumps: Insulin, glucagon, and pramlintide

Some automated insulin delivery systems are able to provide both insulin and glucagon. The benefit of glucagon delivery is that it can help to prevent hypoglycemia by increasing endogenous glucose production. Fast-acting insulin formulations administered by subcutaneous delivery can be not as fast-acting (onset and offset) as the endogenous human insulin produced by human pancreatic islet beta cells. Because of its relative slowness, when a condition of incipient or overt hypoglycemia exists, stoppage of insulin delivery is not perfectly effective, since some of the insulin remains unabsorbed in the subcutaneous space. For this reason, it is effective to deliver glucagon, a very fast-acting drug that is absorbed more quickly than currently-available insulin formulations.

Pramlintide is another drug that may be used in a multi-chamber pump or delivered as a co-formulation along with insulin. Pramlintide causes delayed gastric emptying, and therefore enables the timing for peak carbohydrate absorption to be more closely matched with the slower insulin kinetics. Pramlintide delivered subcutaneously prior to meals can be effective at reducing the postprandial glucose response. The glucose sensing cannula described in this patent can be used to deliver pramlintide as well as insulin or glucagon.

### Subcutaneous Interstitial Continuous Glucose Monitoring (CGM)

CGM is a procedure that provides the patient with diabetes with a subcutaneous interstitial glucose measurements at a high frequency (every several minutes). For example, the hemoglobin A1C (HbA1C), an index of long term glycemic control, is better (lower) in those who use CGM compared to those who do not. As glycemic control improves and HbA1C fall to lower values, the risk for long term complications such as diabetic eye disease, kidney disease, and nerve/foot disease is commensurately lower. Therefore, those who use CGM on a regular basis are at lower risk for these long term complications. Those who use CGM regularly are also at lower risk for short term complications, such as severe hypoglycemia and diabetic ketoacidosis, both of which are common causes of hospitalization and even death.

### Signal Processing to Reduce Error in CGM

A signal processing strategy that can reduce noise in measured CGM data is to use a two-stage Kalman filter comprised of the following components: (1) a delay model to represent the lag between the subcutaneous tissue where the sensing cannula is located and the plasma in the body, (2) a predictive model to forecast future sensor values from past sensor values, and (3) a Kalman filter to adjust sensor readings by optimally combining both the current sensor reading and predictive model estimates of the sensor.

In one example, we can use a simple linear predictive filter given Equation 1 to use past sensor measurements to predict the next sensor measurement. The parameters bo and bi are fit to the past 5 minutes of sensor cannula measurements (i.e. 30 measurements) to predict the next sensor measurement. All measurements from the sensor cannula during this 5-minute time window were used to predict the next sensor measurement.

\vec{g}=b₀+b₁t Equation 1

A Kalman filter is a linear state-space model that operates recursively on noisy input data to produce a statistically optimal estimate of the underlying system state. The general form of the Kalman filter consists of both a transition and observation equation as given in Equation 2, where xₖ and zₖ are the hidden state and observation vectors at time k.

xₖ₊₁=Aₖxₖ+Wₖ

zₖ=Cₖxₖ+vₖ Equation 2

Aₖ and Cₖ are the transition and observation matrices while wₖ and vₖ are Gaussian noise with zero mean. A two-stage Kalman filter can be used to process the sensor cannula data. For stage 1, the Aₖ matrix is the identity matrix, and so the update to the bo and bi parameters amounts to a random walk. The choice of wₖ affects how quickly the slope (b₀) and intercept (b₁) change over time. In this way, in stage 1, a Kalman filter was used to estimate the trend in the sensor data. The 30 prior sensor measurements (5 minutes of data sampled every 10 seconds) was used and a linear regression model was performed to estimate the optimal model parameters which relate the sensor measurement (g) with time (t). The state equation is Equation 2: where bo is the glucose estimate at time to and bi is the rate at which the sensor is changing with respect to time.

For stage 2, the use of the bo and bi parameters determined in stage 1 combined with a 2-compartment interstitial-plasma ODE-based kinetics model. A time lag between interstitial fluid and plasma can be on the order of about 3 to 8 minutes. A time lag between interstitial fluid and plasma can be on the order of about 5-6 minutes. In some instances, the time lag is between about 3 minutes to about 8 minutes. In some instances, the time lag is between about 3 minutes to about 4 minutes, about 3 minutes to about 5 minutes, about 3 minutes to about 6 minutes, about 3 minutes to about 7 minutes, about 3 minutes to about 8 minutes, about 4 minutes to about 5 minutes, about 4 minutes to about 6 minutes, about 4 minutes to about 7 minutes, about 4 minutes to about 8 minutes, about 5 minutes to about 6 minutes, about 5 minutes to about 7 minutes, about 5 minutes to about 8 minutes, about 6 minutes to about 7 minutes, about 6 minutes to about 8 minutes, or about 7 minutes to about 8 minutes. In some instances, the time lag is between about 3 minutes, about 4 minutes, about 5 minutes, about 6 minutes, about 7 minutes, or about 8 minutes. In some instances, the time lag is between at least about 3 minutes, about 4 minutes, about 5 minutes, about 6 minutes, or about 7 minutes. In some instances, the time lag is between at most about 4 minutes, about 5 minutes, about 6 minutes, about 7 minutes, or about 8 minutes. The Aₖ transition matrix accounts for the delay between a plasma compartment and the interstitial compartment, while the Cₖ matrix is the observation model used to predict the next glucose using the regression equation developed from prior measurements. Stage 1 provided smoothing of raw data and stage 2 adjusted for the delay between plasma and interstitium. Alternatively, any ODE-based glucoregulatory model can be used for the stage 2 signal processing, including the more complex model described further below.

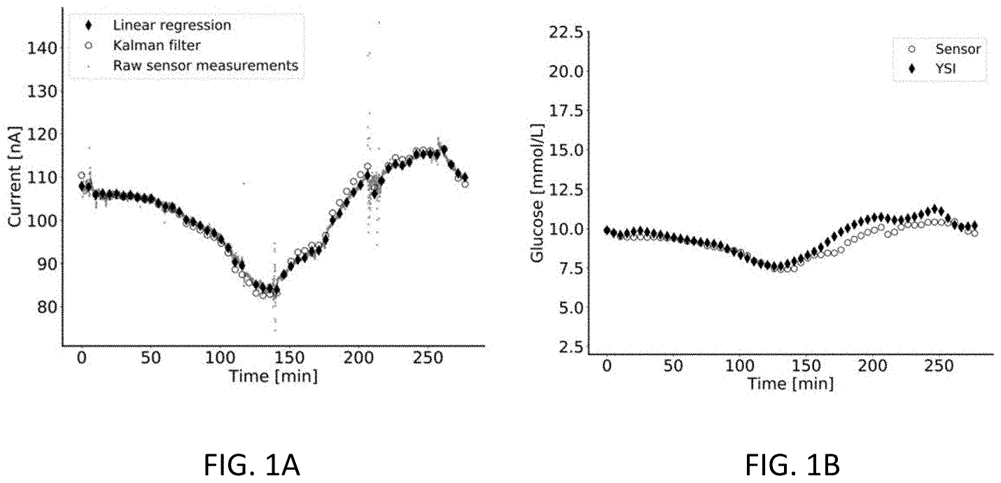

FIG. 1 (top) shows the impact that this two-stage Kalman smoother has on the raw data. The result shows how the calibrated sensor signal fits the YSI data acquired simultaneously during the study (FIG. 1 (bottom)).

### Calibration

Calibration of a glucose sensor involves converting the sensor's measurements in nano Amperes to a glucose measurement in mmol/L (or mM). If one wishes to work in mg/dl units, one can multiply the mM glucose value by 18. Equation 3 can be used to calibrate the sensor,

$$g ( t ) = \frac{i ( t ) - b}{s} \qquad Equation 3$$

whereby s is the sensitivity of the glucose sensor in units of nA/mM, the background current offset (b) is in nA (at zero glucose), g(t) is the glucose in mM at a certain time t, and i(t) is the current in nA at that same time t. In our experiments, for a single-point calibration, the background current was offset (b) to zero, and then solved for sensitivity, s, using the YSI glucose measured at a single time point, g(t), and the current from the sensor measured at that same time point, i(t), such that the sensitivity is the ratio of the sensor current to measured glucose: s =(i(t)-b)/g(t). The sensitivity is then used to convert future measurements from the sensor in nano Amperes (nA) to glucose estimations. The single-point calibration is preferred because it requires only one finger-stick for calibration and it represents the way the sensor will typically be used by a person living with diabetes.

Combining a CGM and an Insulin Cannula in a single Through-the-Skin Tube

At present, many patients who use an on-the-body insulin pump or a tubed pump desire to use CGM. However, in such cases, such patients must place a subcutaneous CGM sensor at a site distant from the pump's insulin cannula, as directed by the user manuals for commercially-available CGM sensors and insulin pumps. Such a requirement can be frustrating because it can be difficult to wear two separate subcutaneous through-the-skin devices. During daily activities, it can be difficult to avoid local trauma to a single through-the skin-device; it is much more difficult to avoid trauma when one is wearing two separately-located through-the-skin devices.

The US issued patent, U.S. Pat. No. 10,780,222, which is hereby incorporated in its entirety, disclosed a dual-function device that allows a CGM to be combined with and collocated with an insulin delivery cannula, thus avoiding the requirement for two through-the-skin devices. It also provides a description of research that determined the cause of interference resulting from locating a conventional, peroxide-measuring sensor very close to the source of delivery of an insulin formulation. The interference did not come from insulin per se but instead from the ubiquitous preservatives present in all insulin formulations, which include phenol and/or cresol, collectively known as phenolics. The main problem causing such interference was the oxidation of the phenolic compounds by a polarizing bias applied to the indicating electrode of over 250-300 mV. When such a polarizing bias is applied to an indicating electrode exposed to a solution that contains the phenolics, two major types of interference develop. First, there is a very large positive-going oxidation artifact that falsely appears to represent a very high glucose level. After the short-lived oxidation peak, the current begins to decline (even when the sensor remains exposed to the phenolic compounds). The current continues to decline until the indicating electrode is permanently unable to respond properly to additional challenges with glucose or other oxidizable compounds. This permanent decay has the (false) appearance of severe hypoglycemia. The cause of this current decay is the oxidation-induced joining together of the many phenolic molecules which leads to a permanent thin layer of polymer on the surface of the indicating electrode. This phenomenon is known as electropolymerization.

The oxidation spike and the subsequent electropolymerization can be avoided by not measuring hydrogen peroxide and instead utilize a redox mediator in a system that is poised at, and can transfer electrons at, a lower bias such as below 200 mV. There are many such redox mediators that can be used effectively for such a sensing system. Redox mediators can include, but are not limited to, Osmium, Ruthenium, Palladium, Platinum, Rhodium, Iridium, Cobalt, Iron, and Copper. There are several effective coordinating ligands that hold the redox mediator in place, such as 4, 4′ dimethyl, 2, 2′ bipyridine and others. Several backbone polymers can be utilized to find the ligands and mediator, including poly N-vinyl imidazole, polyvinyl pyridine, or others.

### Use of a Combined CGM and Insulin Cannula in Patients on CSII

Given the above findings, one can appreciate the benefit of a unified device: more convenience, less pain, better ease of moving about without the risk for local trauma to the insertion site. However, it is also apparent that there needs to be compensation for the phenomenon of dilution artifact, the artifact that perturbs the ability to locally measure glucose in the presence of increasing liquid delivery in the local interstitial space. In the typical use case scenario of SCII with a combined sensing cannula (with the appropriate low bias voltage), there is a risk for the patient to believe that his or her glucose level is low after an insulin bolus is given. In subjects with type 1 diabetes, this dilution artifact can be a function of the liquid in the insulin formulation since such artifact found after saline bolus administration was the of the same magnitude as the artifact after insulin formulation. This study is summarized in more detail below.

Though this dilution artifact is not lengthy (the fluid is absorbed quickly, resolving the dilution artifact), it should not be ignored. In some instances, the direction of the dilution artifact is negative, resulting in an underestimate of the true glucose level. If the reported glucose value is lower than it truly is, a patient may inappropriately reduce insulin delivery, or if the reported glucose value is very low, he may treat himself inappropriately with sugar. In some cases, the artifact leads to elevated glucose readings, but this is an atypical occurrence. If the CGM reading is falsely high, the patient may be tempted to take extra insulin. Therefore, there is a need for the means to appropriately compensate for interstitial dilution artifact.

Procedure for creating CGM sensor and embedding said sensor into the cannula wall

Each cannula contains at least one indicating electrode (the material for which can be gold) and reference electrodes (the material for which can be Ag/AgCl) embedded within the outer wall of the insulin pump cannula. The indicating electrode is covered by glucose oxidase or glucose dehydrogenase and a redox mediator (such as osmium or other suitable redox mediator metal) which is bound by a imidazole-based or pyridine-based ligand, which is in turn bound to a imidazole-based or pyridine-based backbone polymer which conducts the electrons to the indicating electrode where the electrons are donated to the electrode, thus creating a current (amperometric system). It is also possible to completely measure all the available charge, i.e. a coulometric system. A more complete description of one of the methods in which the CGM sensor can be fabricated and built into the cannula wall is given below in Example 1.

The glucoregulatory model can include, but are not limited to, Bergman Minimal Model, UVA Padova Simulator, OHSU Simulator, Dassau-Doyle Model, El-Khatib-Damiano Model, Gani-Reifman Model, Hovorka Model, Bequette Model, Cobelli Model, Cinar Model or Kan Model.

In addition to these glucoregulatory models, machine learning methods can use a neural network approach to predict glucose concentrations. In some embodiments, the machine learning model comprise a neural network.

In addition to glucoregulatory models and neural network approaches, glucose can be predicted by fuzzy logic approaches. A fuzzy logic approach may generally refer to a variable processing method that cam allow multiple possible values to be processed through the same variable. In some cases, using fuzzy logic approaches, multiple solutions are obtained.

Implementation of Interstitial dilution compensation

Shortly after a subcutaneous insulin bolus is given by CSII, there will be error (artifact) in the sensing of glucose by a Combination Insulin Delivery-Glucose Sensing Device (CIDGS device). For this reason, after the bolus, the glucose value as measured by the CIDGS device will not be displayed to the patient. Instead, for a period of time after the bolus (equal to the settling time), forecasted glucose values will be displayed instead. Alternatively, for a period of time after the bolus (equal to the settling time), a combination of the glucose value measured by the CIDGS device and the forecasted glucose values will be displayed. For example, if the CGM in the CIDGS system normally updates the glucose value every 5 minutes, the forecasted glucose value will also be updated every 5 minutes. In some embodiments, the patient will be made aware that the glucose values are forecasted values or the combination of the measured and forecasted values, not measured values. The patient can be made aware by the display of the forecasted value or the combination of the measured and forecasted values by programming a display. In some instances, the display is modified visually. In some examples, the visual modification to the display can comprise, by way of non-limiting example, a different color for the entire display or part of the display, or an added designation (e.g., the letter or a symbol, such as the letter “S”) in part of the display.

A glucoregulatory model, in some cases, will have access to information specific to each patient that increase the likelihood that the forecasted glucose values are accurate. This information includes the amount of insulin-on-board (insulin that has been given but whose effect has not yet occurred), the actual number (or an estimate) of the carbohydrate grams eaten at the meal that accompanies the insulin bolus, and other factors such as whether the patient is exercising or has exercised recently. In some cases, the information is provided by the patient.

Specific details of how a glucoregulatory model can provide forecasted glucose values, including mathematical equations, are provided below in Example 1.

### Specific Method of Overcoming Sensor Error After Insulin Boluses:

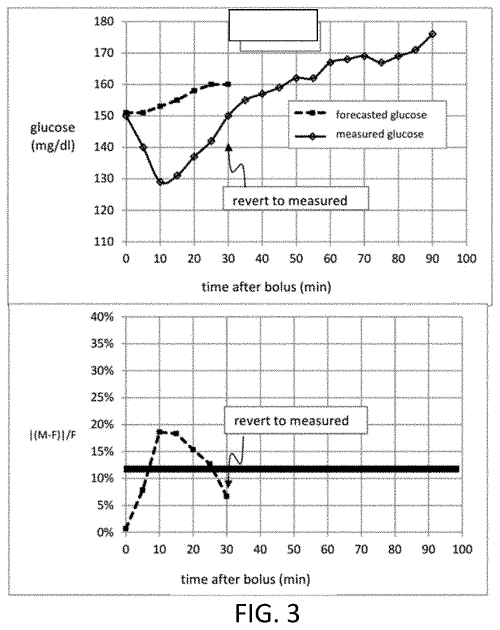

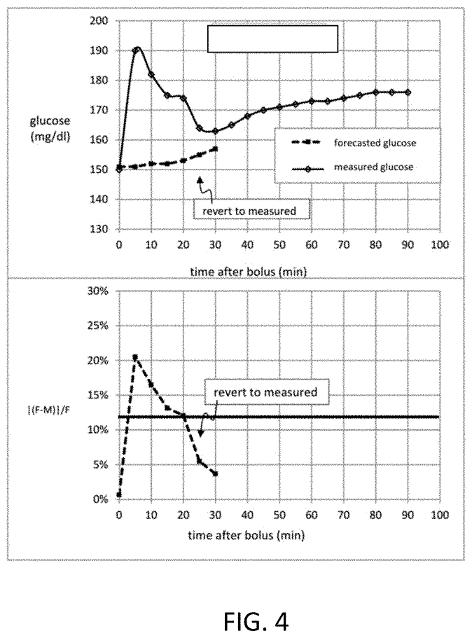

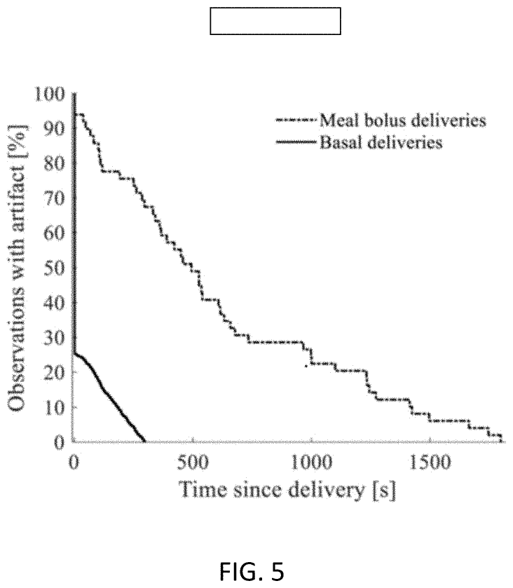

FIGS. 3-4 and the caption for FIGS. 3 and 4 above show the detailed method by which display of erroneous glucose information following an insulin delivery to the patient can be minimized. During the period of dilution artifact, forecasted glucose values are shown in the display rather than the measured glucose values. One very important decision is to decide when the display reverts to showing the measured CGM data rather than forecasted glucose data. It was determined that over 90% of the dilution artifact from larger insulin boluses (i.e. greater than 0.83 units) that are typically given prior to meals disappear after 20-25 minutes as shown in FIG. 5. Immediately after fluid is delivered, most sensor observations exhibit some form of artifact, but the artifact is not present as time passes. Sensor recovery time is shown to be dependent on bolus volume whereby 70-80% of the smaller basal deliveries are free of artifact after 100 seconds while 70-80% of the sensor observations are free of artifact after 10 minutes for larger meal boluses. Whereas smaller doses of insulin (or saline) that are typically given continuously throughout the day (e.g. less than or equal to 0.83 units) had a smaller artifact that disappeared after about 100 seconds. The term that the artifact “disappeared” to mean that the measured sensor reading returns to within 10% of the baseline trend measured prior to the delivery of the insulin (or saline).

The concept of interstitial dilution compensation is that after an insulin delivery is given by the sensing cannula, a prediction algorithm (e.g. a glucoregulatory model or machine learning model) will calculate a forecasted glucose for a window of time during which the dilution artifact is present. The forecasted glucose will be used by the patient and/or by a control algorithm used for automated insulin delivery for a window of time until the dilution artifact is determined to be gone. The algorithm determines how long the forecasted glucose will be used rather than the measured glucose by (1) calculating the forecasted glucose at each time point, (2) measuring the percent difference between the forecasted glucose and the measured glucose, (3) considering the measured glucose variability by calculating the variance of the measured CGM, and (4) determining when the measured glucose falls to within a percentage (e.g. 12% as one example) of the forecasted glucose. Based on our experimental work that has shown that 95% of the sensor dilution artifact is gone after 25 minutes, the maximum duration of the forecast window may be set to 25 minutes for meal bolus insulin deliveries. The use of this percent difference between the forecasted glucose (F) and the measured glucose (M) is demonstrated in FIG. 4. In FIG. 4, we show the metric |M-F|/F*100 can be used to determine if the forecasted glucose (F) deviates by more than 12% (as one example) from the measured glucose, and if it does, then the forecasted glucose is used instead of the measured glucose for up to 25 minutes following a meal bolus insulin delivery. As shown in the lower panel of FIG. 3, this metric is continuously evaluated by the algorithm after an insulin bolus is given. In this example, the algorithm determines that when the metric falls to less than 12% (after initially rising), the displayed glucose value reverts to the measured. This decision criterion can also be set to other levels. The 12% criterion is shown as a black horizontal line in the lower panel of FIG. 3.

To determine when the display reverts to showing the measured CGM data rather than forecasted glucose data, the algorithm uses a metric known as |(M-F)|/F, which means the absolute value of the difference between the measured and forecasted values, divided by the forecasted value, expressed as a percentage.

In another implementation, a Kalman filter, extended Kalman filter, or sigma point Kalman filter can be used to update the forecasted glucose using the measured glucose and to further use the difference between these measurements to update the Kalman gain that determines how much the forecasted vs. the measured glucose is trusted in determining the estimated glucose.

Below is a summary of exemplary steps for performing dilution artifact mitigation:

Step 1, obtain a forecasting algorithm to predict glucose 25-30 minutes in the future.

Train a data-driven forecasting algorithm to generate a predicted glucose value for every time step up to 25-30 minutes in the future (see methods below describing several approaches at doing this).

Alternatively, use an ODE-based model as the forecasting algorithm to predict up to 25-30 minutes in the future by using an ODE solver to solve the ODE system of equations for every time step in the future that is of interest.

Evaluate forecasting algorithm on a held-out test data set collected from unobserved people with type 1 diabetes that was not used for the training of the algorithm.

Ensure that the forecasting algorithm achieves sufficient accuracy in predicting glucose on the held-out test set (e.g. <20 mg/dL RMSE)

Step 2, identify when an insulin bolus has been delivered through the sensing cannula

An insulin bolus may be identified either by notification from an insulin pump or insulin pen when a bolus has been calculated.

An insulin bolus may also be identified automatically by the sensing cannula using a flow sensor.

Step 3, combine the CGM sensor measurement with the CGM estimate from the forecasting algorithm to estimate a final forecasted glucose to display to the user.

Measure CGM using the sensing cannula.

Apply signal processing to the measured CGM using for example a low pass filter that acts to remove noise artifacts from the measured signal by smoothing the signal across a time window. Filters may include, but are not limited to, a low-pass filter, a bandpass filter, and/or a high pass filter.

Use the forecasting algorithm to predict glucose at the current time using the current and previous measured glucose values and insulin deliveries.

Use a Kalman filter to optimally combine the measured glucose and the predicted glucose. The Kalman filter uses a forecasting model along with the measured glucose and an estimate of the measured glucose covariance to generate a forecasted glucose value for the user of the system to view and use. If the variance of the measured CGM is low, then the measured glucose will be weighted more heavily. Whereas if the variance of the measured CGM is high, then the forecasted glucose will be more heavily weighted. The Kalman filter integrates these two glucose estimates to create a forecasted glucose estimate that is a weighted sum of the measured and forecasted glucose estimates.

Repeat above steps for every time step up to 25 minutes after an insulin bolus is delivered.

It is important to note that interstitial dilution compensation is appropriate for open loop control systems in which the patient chooses the size of the bolus, and also for closed loop control systems in which an algorithm determines the size of the bolus. It is also appropriate for hybrid closed loop systems in which the patient must enter certain information, such as the size of each meal bolus.

The algorithm methods described above may be used both following dilution artifacts and also in the case where insulin is not being dose. In other words, the forecasting algorithm may improve the accuracy of any CGM reading, regardless of whether insulin has been dosed recently.

Use of Interstitial Dilution Compensation in MDI treatment

It is also important to note that CSII is not the only use situation in which interstitial dilution compensation avoids CGM error. It is also appropriate for use in the situation in which the patient administers insulin by injection into the subcutaneous tissue in situations in which a CGM is located very close to (for example within 7 mm of) the site of insulin delivered by injection. The situation in which a user injects insulin several times per day is generally called multiple daily injection (MDI) treatment. We have invented a device, known as a CGM Injection Port that allows insulin to be delivered (for example, by an insulin pen) at or very near the indicating electrode of a CGM (see WO/2020/252324 INFUSION DEVICE FOR CONTINUOUS GLUCOSE MONITORING WO-17.12.2020). The use of interstitial dilution compensation avoids CGM error in such a combination device (CGM Injection Port) just as it does in a CIDGS device designed for CSII. The CGM Injection Port is the subject of a separate patent application.

### Other Embodiments:

In one embodiment, the disclosure provides diabetes management device, including a glucose sensor implanted under the skin. The diabetes management device includes a signal processing algorithm to improve the accuracy of the glucose sensor measurement. The diabetes management device further includes a signal processing algorithm that includes a model to predict glucose.

In some embodiments, the model to predict glucose is one or more of the following: linear regression model, support vector regression model, multivariable adaptive regressive spline model, neural network model, long short term memory (LSTM) neural network, ridge regression model, Lasso regression model, ElasticNet regression model, or an ODE-based glucoregulatory model comprised of one or more compartments that represent insulin kinetics and dynamics, glucagon kinetics and dynamics, carbohydrate kinetics and dynamics, pramlintide kinetics and dynamics.

In some embodiments, the glucose measurement device is located near a subcutaneous insulin delivery site. In some cases, said diabetes management device includes an interstitial dilution compensation algorithm which manages the presentation of glucose to a user or to an automated hormone delivery algorithm during a period of time following delivery of the hormone for the purpose of mitigating the impact of a dilution artifact on the sensor. In some cases, said dilution compensation algorithm uses a predictive model to calculate the glucose presented to the person or automated hormone delivery algorithm.

In some embodiments, the predictive model is an ODE-based glucoregulatory model comprised of one or more compartments representing insulin kinetics and dynamics, glucagon kinetics and dynamics, carbohydrate kinetics and dynamics, pramlintide kinetics and dynamics.

In some embodiments, the predictive model is one or more of the following: long-short-term-memory (LSTM) neural network, random forest, linear regression model, support vector regression model, multivariable adaptive regressive spline model, neural network model, ridge regression model, Lasso regression model, ElasticNet regression model.

In some embodiments, the predicted glucose is determined using a Kalman filter, extended Kalman filter, or sigma point Kalman filter.

In some embodiments, the predicted glucose is determined using a Kalman filter, extended Kalman filter, or sigma point Kalman filter.

In some embodiments, the period of time following hormone delivery is determined in real-time based on the difference between the glucose measured by the sensor and the glucose predicted by the model.

In some embodiments, pramlintide is an additional hormone delivered by the device.

In some embodiments, the glucose measurements are filtered to remove noise.

In some embodiments, the predictive model is used to improve the accuracy of the glucose measurement.

In some embodiments, the predictive model is used to improve the accuracy of the glucose measurement.

In some embodiments, insulin is delivered by a tubed insulin pump or patch insulin pump using an open loop insulin delivery system. In some embodiments, insulin is delivered by a tubed insulin pump or patch insulin pump using a closed loop insulin delivery system. In some embodiments, insulin is delivered by a tubed insulin pump or patch insulin pump using a hybrid closed loop insulin delivery system.

In some embodiments, insulin is delivered via a subcutaneous port or directly into the subcutaneous space by injection. In some embodiments, glucagon is also delivered by the device.

### Computing System

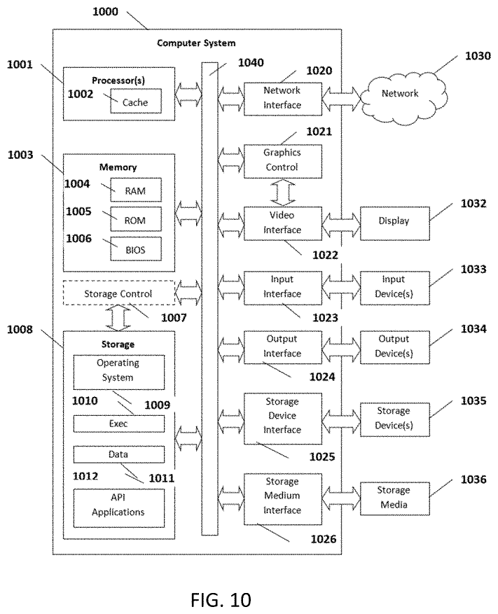

Referring to FIG. 10, a block diagram is shown depicting an exemplary machine that includes a computer system 1000 (e.g., a processing or computing system) within which a set of instructions can execute for causing a device to perform or execute any one or more of the aspects and/or methodologies for static code scheduling of the present disclosure. The components in FIG. 10 are examples only and do not limit the scope of use or functionality of any hardware, software, embedded logic component, or a combination of two or more such components implementing particular embodiments.

Computer system 1000 may include one or more processors 1001, a memory 1003, and a storage 1008 that communicate with each other, and with other components, via a bus 1040. The bus 1040 may also link a display 1032, one or more input devices 1033 (which may, for example, include a keypad, a keyboard, a mouse, a stylus, etc.), one or more output devices 1034, one or more storage devices 1035, and various tangible storage media 1036. All of these elements may interface directly or via one or more interfaces or adaptors to the bus 1040. For instance, the various tangible storage media 1036 can interface with the bus 1040 via storage medium interface 1026. Computer system 1000 may have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

Computer system 1000 includes one or more processor(s) 1001 (e.g., central processing units (CPUs), general purpose graphics processing units (GPGPUs), or quantum processing units (QPUs)) that carry out functions. Processor(s) 1001 optionally contains a cache memory unit 1002 for temporary local storage of instructions, data, or computer addresses. Processor(s) 1001 are configured to assist in execution of computer readable instructions. Computer system 1000 may provide functionality for the components depicted in FIG. 10 as a result of the processor(s) 1001 executing non-transitory, processor-executable instructions embodied in one or more tangible computer-readable storage media, such as memory 1003, storage 1008, storage devices 1035, and/or storage medium 1036. The computer-readable media may store software that implements particular embodiments, and processor(s) 1001 may execute the software. Memory 1003 may read the software from one or more other computer-readable media (such as mass storage device(s) 1035, 1036) or from one or more other sources through a suitable interface, such as network interface 1020. The software may cause processor(s) 1001 to carry out one or more processes or one or more steps of one or more processes described or illustrated herein. Carrying out such processes or steps may include defining data structures stored in memory 1003 and modifying the data structures as directed by the software.

The memory 1003 may include various components (e.g., machine readable media) including, but not limited to, a random access memory component (e.g., RAM 1004) (e.g., static RAM (SRAM), dynamic RAM (DRAM), ferroelectric random access memory (FRAM), phase-change random access memory (PRAM), etc.), a read-only memory component (e.g., ROM 1005), and any combinations thereof. ROM 1005 may act to communicate data and instructions unidirectionally to processor(s) 1001, and RAM 1004 may act to communicate data and instructions bidirectionally with processor(s) 1001. ROM 1005 and RAM 1004 may include any suitable tangible computer-readable media described below. In one example, a basic input/output system 1006 (BIOS), including basic routines that help to transfer information between elements within computer system 1000, such as during start-up, may be stored in the memory 1003.

Fixed storage 1008 is connected bidirectionally to processor(s) 1001, optionally through storage control unit 1007. Fixed storage 1008 provides additional data storage capacity and may also include any suitable tangible computer-readable media described herein. Storage 1008 may be used to store operating system 1009, executable(s) 1010, data 1011, applications 1012 (application programs), and the like. Storage 1008 can also include an optical disk drive, a solid-state memory device (e.g., flash-based systems), or a combination of any of the above. Information in storage 1008 may, in appropriate cases, be incorporated as virtual memory in memory 1003.

In one example, storage device(s) 1035 may be removably interfaced with computer system 1000 (e.g., via an external port connector (not shown)) via a storage device interface 1025. Particularly, storage device(s) 1035 and an associated machine-readable medium may provide non-volatile and/or volatile storage of machine-readable instructions, data structures, program modules, and/or other data for the computer system 1000. In one example, software may reside, completely or partially, within a machine-readable medium on storage device(s) 1035. In another example, software may reside, completely or partially, within processor(s) 1001.

Bus 1040 connects a wide variety of subsystems. Herein, reference to a bus may encompass one or more digital signal lines serving a common function, where appropriate. Bus 1040 may be any of several types of bus structures including, but not limited to, a memory bus, a memory controller, a peripheral bus, a local bus, and any combinations thereof, using any of a variety of bus architectures. As an example and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 1000 may also include an input device 1033. In one example, a user of computer system 1000 may enter commands and/or other information into computer system 1000 via input device(s) 1033. Examples of an input device(s) 1033 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. In some embodiments, the input device is a Kinect, Leap Motion, or the like. Input device(s) 1033 may be interfaced to bus 1040 via any of a variety of input interfaces 1023 (e.g., input interface 1023) including, but not limited to, serial, parallel, game port, USB, FIREWIRE, THUNDERBOLT, or any combination of the above.

In particular embodiments, when computer system 1000 is connected to network 1030, computer system 1000 may communicate with other devices, specifically mobile devices and enterprise systems, distributed computing systems, cloud storage systems, cloud computing systems, and the like, connected to network 1030. Communications to and from computer system 1000 may be sent through network interface 1020. For example, network interface 1020 may receive incoming communications (such as requests or responses from other devices) in the form of one or more packets (such as Internet Protocol (IP) packets) from network 1030, and computer system 1000 may store the incoming communications in memory 1003 for processing. Computer system 1000 may similarly store outgoing communications (such as requests or responses to other devices) in the form of one or more packets in memory 1003 and communicated to network 1030 from network interface 1020. Processor(s) 1001 may access these communication packets stored in memory 1003 for processing.

Examples of the network interface 1020 include, but are not limited to, a network interface card, a modem, and any combination thereof. Examples of a network 1030 or network segment 1030 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 1030, may employ a wired and/or a wireless mode of communication. In general, any network topology may be used.

Information and data can be displayed through a display 1032. Examples of a display 1032 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 1032 can interface to the processor(s) 1001, memory 1003, and fixed storage 1008, as well as other devices, such as input device(s) 1033, via the bus 1040. The display 1032 is linked to the bus 1040 via a video interface 1022, and transport of data between the display 1032 and the bus 1040 can be controlled via the graphics control 1021. In some embodiments, the display is a video projector. In some embodiments, the display is a head-mounted display (HIVID) such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In addition to a display 1032, computer system 1000 may include one or more other peripheral output devices 134 including, but not limited to, an audio speaker, a printer, a storage device, and any combinations thereof. Such peripheral output devices may be connected to the bus 1040 via an output interface 1024. Examples of an output interface 1024 include, but are not limited to, a serial port, a parallel connection, a USB port, a FIREWIRE port, a THUNDERBOLT port, and any combinations thereof.

In addition or as an alternative, computer system 1000 may provide functionality as a result of logic hardwired or otherwise embodied in a circuit, which may operate in place of or together with software to execute one or more processes or one or more steps of one or more processes described or illustrated herein. Reference to software in this disclosure may encompass logic, and reference to logic may encompass software. Moreover, reference to a computer-readable medium may encompass a circuit (such as an IC) storing software for execution, a circuit embodying logic for execution, or both, where appropriate. The present disclosure encompasses any suitable combination of hardware, software, or both.

Those of skill in the art will appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module executed by one or more processor(s), or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In accordance with the description herein, suitable computing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will also recognize that select televisions, video players, and digital music players with optional computer network connectivity are suitable for use in the system described herein. Suitable tablet computers, in various embodiments, include those with booklet, slate, and convertible configurations, known to those of skill in the art.

In some embodiments, the computing device includes an operating system configured to perform executable instructions. The operating system is, for example, software, including programs and data, which manages the device's hardware and provides services for execution of applications.

Those of skill in the art will recognize that suitable server operating systems include, by way of non-limiting examples, FreeB SD, OpenBSD, NetB SD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smartphone operating systems include, by way of non-limiting examples, Nokia® Symbian° OS, Apple® iOS®, Research In Motion® BlackBerry OS®, Google° Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3®, Sony® PS4®, Microsoft® Xbox 360®, Microsoft Xbox One, Nintendo® Wii®, Nintendo® Wii U®, and Ouya®.

### Non-Transitory Computer Readable Storage Medium

In some embodiments, the platforms, systems, media, and methods disclosed herein include one or more non-transitory computer readable storage media encoded with a program including instructions executable by the operating system of an optionally networked computing device. In further embodiments, a computer readable storage medium is a tangible component of a computing device. In still further embodiments, a computer readable storage medium is optionally removable from a computing device. In some embodiments, a computer readable storage medium includes, by way of non-limiting examples, CD-ROMs, DVDs, flash memory devices, solid state memory, magnetic disk drives, magnetic tape drives, optical disk drives, distributed computing systems including cloud computing systems and services, and the like. In some cases, the program and instructions are permanently, substantially permanently, semi-permanently, or non-transitorily encoded on the media.

Computer program

In some embodiments, the platforms, systems, media, and methods disclosed herein include at least one computer program, or use of the same. A computer program includes a sequence of instructions, executable by one or more processor(s) of the computing device's CPU, written to perform a specified task. Computer readable instructions may be implemented as program modules, such as functions, objects, Application Programming Interfaces (APIs), computing data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as ^{Microsoft}® .NET or Ruby on Rails (RoR). In some embodiments, a web application utilizes one or more database systems including, by way of non-limiting examples, relational, non-relational, object oriented, associative, XML, and document oriented database systems. In further embodiments, suitable relational database systems include, by way of non-limiting examples, Microsoft° SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous JavaScript and XML (AJAX), Flash® ActionScript, JavaScript, or Silverlight. In some embodiments, a web application is written to some extent in a server-side coding language such as Active Server Pages (ASP), ColdFusion , Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as ^{IBM}® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

### Mobile Application

In some embodiments, a computer program includes a mobile application provided to a mobile computing device. In some embodiments, the mobile application is provided to a mobile computing device at the time it is manufactured. In other embodiments, the mobile application is provided to a mobile computing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, JavaScript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple° App Store, Google° Play, Chrome WebStore, BlackBerry° App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Web Browser Plug-In

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB .NET, or combinations thereof.

Web browsers (also called Internet browsers) are software applications, designed for use with network-connected computing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called microbrowsers, mini-browsers, and wireless browsers) are designed for use on mobile computing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony PSP™ browser.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, a distributed computing resource, a cloud computing resource, or combinations thereof. In further various embodiments, a software module comprises a plurality of files, a plurality of sections of code, a plurality of programming objects, a plurality of programming structures, a plurality of distributed computing resources, a plurality of cloud computing resources, or combinations thereof. In various embodiments, the one or more software modules comprise, by way of non-limiting examples, a web application, a mobile application, a standalone application, and a distributed or cloud computing application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on a distributed computing platform such as a cloud computing platform. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for storage and retrieval of glucose measurement information. In various embodiments, suitable databases include, by way of non-limiting examples, relational databases, non-relational databases, object oriented databases, object databases, entity-relationship model databases, associative databases, XML databases, document oriented databases, and graph databases. Further non-limiting examples include SQL, PostgreSQL, MySQL, Oracle, DB2, Sybase, and MongoDB. In some embodiments, a database is Internet-based. In further embodiments, a database is web-based. In still further embodiments, a database is cloud computing-based. In a particular embodiment, a database is a distributed database. In other embodiments, a database is based on one or more local computer storage devices

### EXAMPLES

### Example 1

### Procedure for Fabricating a CGM sensor Embedded Within the Wall of a Polymeric Cannula.

Prior to their separation into individual sensing units, sensors were fabricated into planar electrode arrays with multiple sensor units per array. Each sensor unit was comprised of a silver reference electrode and 1-3 gold indicating electrodes. A sputter chamber was used to apply the metals to a flexible polymer sheet such as polyimide. Other types of polymers can be used instead of polyimide. Patterns and shapes of the electrodes were created using photolithography techniques and microablation using an infrared wavelength (1064 nm) laser tool. The silver electrode was converted to a Ag/AgCl combined counter/reference electrode by exposing the silver to ferric chloride. In another embodiment, there is a gold or platinum counter electrode in addition to an indicating electrode and a reference electrode. The individual sensor units were separated from the array using an ultraviolet wavelength (355 nm) laser tool. The flexible individual sensor units were then laminated to the surface of a 25 gauge stainless steel tube using epoxy cement. In some embodiments, the metal tube is omitted. The sensing cannula is built so that the depth of insulin delivery beneath skin was approximately 5-8 mm. A skin-worn battery-powered electronic module was developed to (1) provide the bias potential to the sensor, (2) convert the current generated by the glucose sensor to a proportional voltage using a transimpedance amplifier, and (3) sample and transmit the glucose sensor data wirelessly to a computing device such as a smartphone or laptop using a micro-controller and a Bluetooth Low Energy radio transceiver chip (NRF-51822; Nordic Inc).

### Sensing Cannula Electrochemistry

The chemistry of the redox-mediated sensor, included osmium coordinated within a ligand, is followed by binding the osmium/ligand complex to a polymer chain. As a redox mediator, osmium was used because it is suitable for accepting electrons from glucose oxidase, more specifically from the prosthetic group of glucose oxidase (flavin adenine dinucleotide, FAD). Other metals such as ruthenium, tantalum, iron and others can also be used as redox mediators. After the osmium-ligand complex was formed, this complex was bound to a polymer backbone, poly(N-vinyl imidazole, PVI) (BOC Sciences). Once the osmium ligand complex is bound to PVI, the entire complex is bound to a gold electrode poised at 175-180 mV vs Ag/AgCl reference electrode. The bias can also be poised much lower, though such a lower bias results in generation of a lower glucose-induced current. The electrons originated from glucose are transported to the gold electrode on which the osmium-ligand-backbone is deposited, thus creating a glucose-induced current that is not adversely affected by excipients in commercial insulin formulations. As the electrons are donated to the gold electrode, the osmium mediator switches back to its oxidized state and can then acquire more electrons from the FAD group of the glucose oxidase when glucose is present. Through cyclic voltammetry experiments, we found that the maximal current of the sensor when exposed to glucose was at 175 mV. And, at that voltage, there was no interference from phenol, meta-cresol, acetaminophen, ascorbic acid and uric acid. Is some cases, lower bias voltages may be used with no ill sensing effects.

### Preparation of Osmium-Ligand Complex and Osmium-Ligand-Backbone Complex

One molar part of K2OsCl6 is dissolved in ethylene glycol and 2.5-3 M parts of 4,4′ dimethyl 2,2′ bipyridine are added. The solution is refluxed for 12-24 h in a fume hood at atmospheric pressure and at a temperature of 200° C. In terms of the reflux apparatus procedure, the key substrates stay in the lower spherical part of the apparatus. In contrast, since the temperature is kept at or slightly above the boiling point of the solvent, some of the solvent evaporates into the vertical tubular element, then immediately condenses and returns to the lower part. Since the temperature of a boiling solution remains at the boiling point even when excess heat is applied, the use of the reflux condensing procedure is a convenient way to continuously keep a solution at a very specific temperature (based on the choice of solvent) for a prolonged period of time.

After evaporation of the ethylene glycol, the solid osmium bound to the 4,4′ dimethyl 2,2′ bipyridine is reacted with poly(N-vinyl imidazole, PVI) in the same way as described above, i.e. using reflux condensing held at 200 deg C in ethylene glycol for 12-24 h. The molar ratios can be calculated using the average molecular weight of the particular batch of PVI and the fact that the desired ratio is one osmium-ligand complex per every 6-10 repeating vinyl imidazole units. The ethylene glycol is removed (evaporated), leaving the osmium-ligand-PVI complex as a solid brown material. This material is then mixed with glucose oxidase (Sigma-Aldrich) at a temperature of about 35-45 deg C, avoiding higher temperatures that could denature the glucose oxidase. This mixture, applied on a clean gold electrode, is then exposed to glutaraldehyde (Sigma-Aldrich) vapor in a fume hood for 1-2 h (glutaraldehyde has a very high vapor pressure and is probably toxic, thus exposure to humans must be avoided). The glutaraldehyde crosslinks the glucose oxidase and thus avoids leaching of the enzyme into mammalian tissue (glucose oxidase is not a mammalian protein and probably can cause serious allergic reactions in mammals). The electrode, now coated with glucose oxidase and osmium-ligand-PVI backbone, is then coated with an outer mem-brane of polyurethane as follows: An outer membrane by dip coating into a solvated blend of polyvinylpyridine-co-styrene (PVP-co-S) with commercially available Pellethane and a silicone containing poly urethane. The polymer blend admixed in 1:1 dimethylacetamide (DMAc) and tertrahydrofuran. Dip coating results in a coating deposited over the top of the glucose oxidase/osmium-ligand/polymer backbone. After the polyurethane-solvent solution is coated on to the glucose oxidase/osmium-ligand/polymer backbone, the solvent completely evaporates, leaving behind a pure film of polyurethane, coating the other layers.

The indicating electrode coated as described above is used either in conjunction with a reference electrode or a combined counter/reference electrode. For the purposes of this disclosure, both configuration will be referred to as a reference electrode.

Studies with such combined sensor plus cannula devices were carried out in swine and in humans with Type 1 diabetes. These studies showed that both the initial oxidation spike and the subsequent electropolymerization-induced current decay were absent with the low-bias redox mediator type of CGM sensor was incorporated into an insulin delivery cannula. However, when a standard platinum-based sensor polarized at a bias of over 400 mV and designed to measure hydrogen peroxide was used, there was both a large positive-going oxidative artifact and a subsequent current decay caused by oxidative electropolymerization that led to permanent electrode failure.

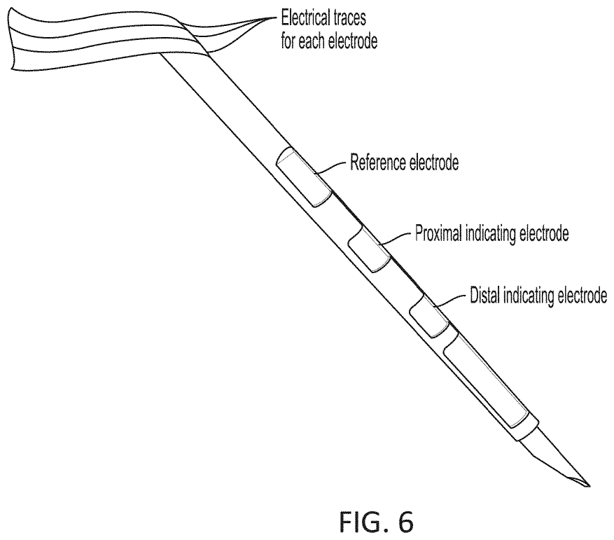

FIG. 6 shows a fabricated glucose sensing cannula designed using the methods described above. The tube is sharp and metallic in this FIG. 6, but it should be noted that embodiments also exist in which the tube is polymeric and flexible and in which the distal end of the tube is not sharp. In the latter case, a removable sharp central stylet or a removable sharp peripheral trocar can be used to introduce the sensing cannula.

Specific Use of a Glucoregulatory Model to generate Forecasted Glucose Values:

Specific details of how forecasted glucose values can be calculated (using a model) in order to provide glucose values to the patient during the period after a bolus of insulin during which dilution artifact makes it unwise to display the values measured by the CGM in a combination (CIDGS) device. This model consists of ordinary differential equations that are used to model the insulin kinetics model based on Kobayashi et al, insulin dynamics model, and a glucose kinetics model. This model has been used in both an in silico simulator of metabolism and the model has also been used to forecast glucose several hours in the future within a model predictive control (MPC) control algorithm.

The glucoregulatory model utilizes the following equations:

$$( t ) = - ( \text{?} + {k}_{21} + X ( \text{?} ) ) \text{?}$$

$${\dot{Q}}_{2} ( t ) = {k}_{21} {Q}_{1} ( t ) - {k}_{12} {Q}_{2}$$

$$\dot{X} ( t ) = - {P}_{2} X ( t ) + {P}_{3} ( I ( t ) - {I}_{b} )$$

$$\text{?} ( t ) = - {k}_{a} \text{?}$$

$$\dot{I} ( t ) = - \text{?} I ( t ) + \text{?}$$

$${U}_{G} = \text{?}$$

$$\text{?} \text{indicates text missing or illegible when filed}$$

where Q₁ and Q₂ are glucose masses in accessible and nonaccessible compartments, respectively (mg/kg). The Q₁ compartment is the glucose in plasma where it can be measured (i.e. accessible), while the Q₂ compartment is the glucose in tissue where it cannot be measured (i.e. nonaccessible). Pi is the glucose effectiveness (min⁻¹). X is the effect of insulin on blood glucose (min⁻¹). P₂ represents the decay rate of X (min⁻¹). P₃ shows the effective rate of insulin in plasma (min⁻² per mU/L). The ratio between P₃ (min⁻² per mU/L) and P₂ (min⁻¹) represents the interstitial fluid (1SF). Q_{1b} and 1b are basal plasma glucose and insulin, respectively. k₁₂ and k₂₁ (min⁻¹) are rate parameters describing glucose exchange kinetics, respectively. X₁ (t) is the amount of insulin in the subcutaneous depot (mU/kg), 1(t) is the plasma insulin concentration (mU/L), and u(t) is subcutaneous infused insulin (mU/kg/min). ke is the elimination rate of insulin (min⁻¹), ka is the absorption rate of insulin (min⁻¹), Vd is the insulin volume of distribution (L/kg), and τ is the time delay for injected insulin to be effective in the interstitial fluid (min), which was set to zero. UG represents the glucose absorption rate from meals (mg/kg/min). t_{max,G} (min) is the time-to-maximum appearance rate of glucose in Q₁, A_{G} is the carbohydrate bioavailability (unitless), to is the meal announcement time (min), and D_{G} is the estimated carbohydrate intake (mg/kg). D_{G} is converted from grams to milligrams per kilograms to be compatible with Q₁ in the glucose kinetics model. The process model determines the forecasted glucose level at any time over the prediction horizon.

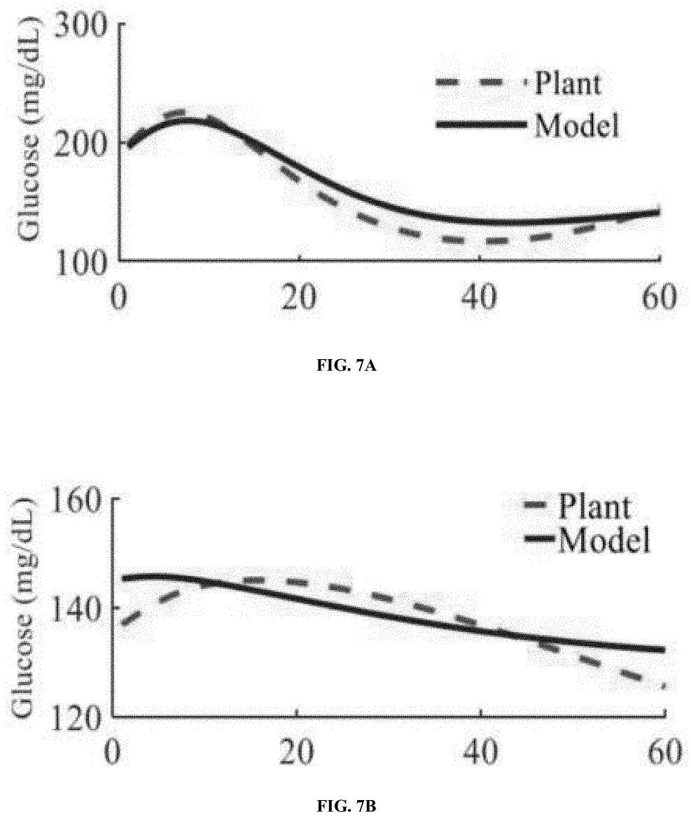

FIGS. 7A-7B shows how the ODE model forecasts glucose and how it compares to an actual human (labeled here as a plant). The example in FIG. 7A shows a markedly hyperglycemic individual and the example in FIG. 7B shows mild hyperglycemia. The model takes into consideration how much insulin has been given to the patient. It is clear from these graphs that this glucoregulatory model can estimate the true glucose in humans with type 1 diabetes with reasonable accuracy. These data are taken from Resalat, N. et al, “Adaptive Control of an Artificial Pancreas using Model Identification, Adaptive Postprandial Insulin Delivery and Exercise,” Journal of Diabetes Science and Technology, vol. 13, pp. 1044-1053, 2019, incorporated by reference in its entirety herein.

### Specific use of an LSTM Neural Network to Forecast Glucose Values

The LSTM forecasting algorithm can be used as the forecasting algorithm to mitigate the dilution artifact and improve accuracy of the measured sensor.

In addition to using an ODE-based physical model to forecast glucose values, a black-box model such as a neural network model may be used to forecast glucose using features as inputs to the model. For this model, the input features to the LSTM included CGM and insulin for 3 hours prior to a prediction. Insulin on board was the amount of insulin estimated to be in the blood of the person with diabetes and was calculated using the equation below whereby the variable B are insulin boluses given at time k. The time constant decay of insulin in the blood is specified by zIOB with a value of 0.012. Each time index j is a 5-minute time point.

$$\text{?}$$

$$\text{?} \text{indicates text missing or illegible when filed}$$

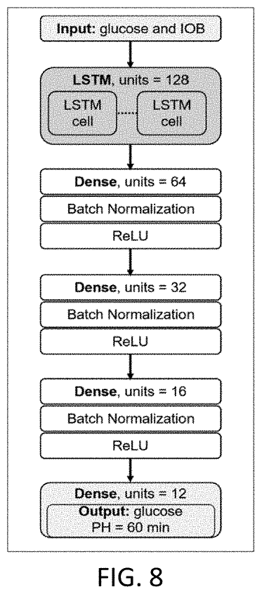

The LSTM architecture is provided in FIG. 8:

A machine learning model such as an LSTM model needs to be trained. A data set provided by the Tidepool Big Data Donation Dataset (Tidepool, Palo Alto, Calif.) to train the LSTM model using cross-validation. The model was trained on 175 people with type 1 diabetes across 41,318 days. Evaluation of the accuracy of the forecasting algorithm was done on 75 new people that were not included in the training data set including 11,333 new days of time-matched CGM and insulin data. In addition to training a single population LSTM model on all of the data, we also trained separate models on different regions of CGM values to determine if a model trained only on a specific range of starting CGM values may perform better than a population model trained on all glucose.

The performance of the LSTM was compared with other data-driven models including a ridge linear regression algorithm and a random forest. The random forest was designed with 100 trees and a maximum tree depth of 16. These machine learning algorithms were compared with two naive estimates of glucose forecasting: (1) a simple zero-order hold which presumes that the glucose will not change in the future, and (2) a simple linear regression fit to the prior 10 minutes of CGM data and extended into the forecasting window to estimate future glucose values. To compare the performance, we used root mean squared error (RMSE), mean absolute error (MAE), and mean error (ME). The equations for these metrics are given below.

$$\text{?}$$

$$\text{?} \text{indicates text missing or illegible when filed}$$

Comparisons of the prediction accuracy with a 30-minute horizon and a 60-minute horizon are given in the table below. We can see here that the LSTM population model had the highest accuracy at 30-minute predictions with an RMSE of 19.8±3.2 mg/dL, however other forecasting models like the random forest and linear ridge regression also performed well. Designing specific LSTM models for certain glucose ranges as shown in the LSTM cluster results, did not result in significant improvements in forecasting overall. The naive estimators were significantly worse than the machine learning algorithms.

TABLE 1
Performance of LSTM population and cluster-based models in forecasting glucose
compared with naive predictors and also other data-drive forecasting methods.

| | | 30 minutes | | | 60 minutes | | |
|---|---|---|---|---|---|---|---|
| Predicition horizon | | RMSE | MAE | ME | RMSE | MAE | ME |
| Model | Range mg/dL | MEAN ± STD mg/dL | | | | | |
| Zero-order hold | Overall | 25.4 ± 4.6 | 18.3 ± 3.4 | 0.0 ± 0.4 | 39.8 ± 7.0 | 29.2 ± 5.2 | 0.0 ± 0.7 |
| | <70 | 28.1 ± 7.8 | 21.6 ± 6.4 | 19.9 ± 6.9 | 50.6 ± 11.8 | 40.5 ± 10.7 | 39.5 ± 11.2 |
| | 70-180 | 22.9 ± 3.8 | 16.6 ± 2.9 | 1.7 ± 1.3 | 34.3 ± 5.2 | 25.4 ± 3.9 | 4.8 ± 3.3 |
| | >180 | 34.8 ± 5.9 | 26.2 ± 4.7 | −11.5 ± 5.3 | 58.5 ± 8.9 | 45.9 ± 8.0 | −28.7 ± 11.2 |
| 10-min linear | Overall | 30.6 ± 5.7 | 21.4 ± 4.2 | 0.0 ± 0.1 | 65.7 ± 12.4 | 46.7 ± 9.3 | −0.1 ± 0.2 |
| trend | <70 | 28.8 ± 7.6 | 20.5 ± 5.6 | −2.2 ± 3.9 | 67.9 ± 20.3 | 47.6 ± 15.0 | 14.0 ± 14.2 |
| | 70-180 | 29.0 ± 5.7 | 20.1 ± 4.2 | −13 ± 0.8 | 61.4 ± 11.6 | 43.3 ± 8.7 | −1.2 ± 1.2 |
| | >180 | 37.1 ± 6.5 | 27.5 ± 5.1 | 6.1 ± 2.0 | 81.9 ± 15.4 | 62.4 ± 12.7 | 0.6 ± 6.5 |
| Linear regression | Overall | 21.4 ± 3.6 | 15.4 ± 2.7 | −1.2 ± 2.0 | 35.2 ± 6.0 | 26.2 ± 4.3 | −1.3 ± 4.7 |
| | <70 | 24.7 ± 5.2 | 19.7 ± 4.0 | 18.3 ± 3.9 | 51.2 ± 8.7 | 45.8 ± 7.8 | 6.3 ± 7.8 |
| | 70-180 | 19.0 ± 3.2 | 13.7 ± 2.3 | 1.4 ± 0.4 | 27.6 ± 3.7 | 21.1 ± 2.8 | 6.3 ± 1.3 |
| | >180 | 29.4 ± 4.0 | 22.2 ± 3.1 | −15.4 ± 2.0 | 55.9 ± 69 | 45.1 ± 6.1 | −41.0 ± 6.1 |
| Random forest | Overall | 20.8 ± 3.4 | 14.8 ± 2.5 | −1.5 ± 1.7 | 14.2 ± 5.7 | 25.0 ± 4.2 | −2.0 ± 4.4 |
| | <70 | 25.3 ± 4.9 | 21.7 ± 3.9 | 21.7 ± 1.9 | 49.5 ± 8.2 | 45.1 ± 7.1 | 45.1 ± 7.1 |
| | 70-180 | 18.4 ± 2.8 | 12.1 ± 2.1 | 0.1 ± 0.6 | 38.8 ± 34 | 20.1 ± 2.6 | 3.9 ± 1.6 |
| | >180 | 28.5 ± 4.4 | 21.2 ± 3.5 | −12.1 ± 2.5 | 54.3 ± 7.3 | 43.1 ± 6.7 | −36.0 ± 7.0 |
| LSTM | Overall | 19.8 ± 3.2 | 14.2 ± 2.3 | −1.8 ± 2.4 | 33.2 ± 5.4 | 24.5 ± 3.9 | −0.4 ± 4.9 |
| (population) | <70 | 25.4 ± 4.3 | 22.3 ± 3.6 | 22.3 ± 3.6 | 49.7 ± 8.0 | 45.8 ± 7.1 | 45.8 ± 7.1 |
| | 70-180 | 17.0 ± 2.4 | 12.3 ± 1.8 | −0.4 ± 1.2 | 26.2 ± 3.2 | 19.7 ± 2.5 | 4.9 ± 2.5 |
| | >180 | 28.0 ± 4.0 | 21.0 ± 3.2 | −12.9 ± 2.6 | 51.8 ± 6.7 | 41.2 ± 6.2 | −32.3 ± 6.8 |
| LSTM | Overall | 19.8 ± 3.2 | 14.3 ± 2.4 | −0.6 ± 2.7 | 33.2 ± 5.4 | 24.8 ± 4.0 | 1.5 ± 5.8 |
| (cluster-based) | <70 | 28.0 ± 4.8 | 24.7 ± 4.2 | 24.7 ± 4.2 | 53.9 ± 8.0 | 50.1 ± 7.3 | 50.1 ± 7.3 |
| | 70-180 | 17.1 ± 2.6 | 12.4 ± 2.0 | 1.5 ± 1.4 | 26.4 ± 1.4 | 20.1 ± 2.8 | 7.6 ± 3.1 |
| | >180 | 27.4 ± 3.6 | 20.8 ± 2.8 | −14.4 ± 2.1 | 50.2 ± 6.1 | 39.9 ± 5.5 | −33.7 ± 5.7 |

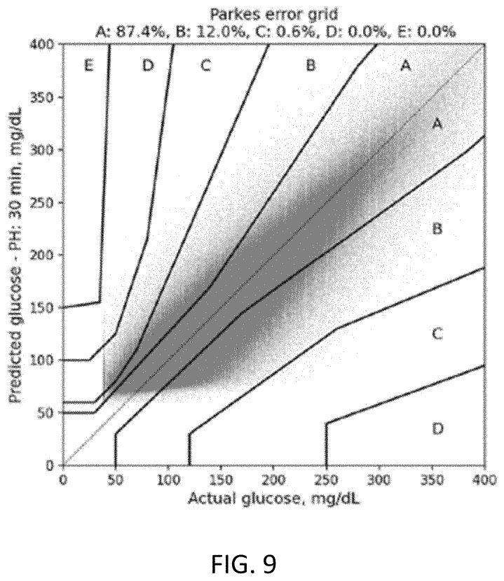

In FIG. 9, we can see a Parkes Error Grid

### Example 2

### Insulin-Induced Dilution of the Interstitial Space—Clinical Study in Patients with Type 1 Diabetes

### Quantifying Sensor Artifact and Mitigating the Effect of Interstitial Dilution

At Oregon Health and Science University (OHSU), a study of 7 persons with Type 1 diabetes was carried out to address the feasibility of using a combined CGM glucose sensor and insulin delivery cannula in persons using a tubed insulin pump. The primary objective of this study was to compare the artifact (error) caused by the delivery of either insulin analog vs. saline control solution, both delivered from the sensing cannula.

After deliveries of each bolus of insulin aspart (Novo-Nordisk), the glucose measurement artifact caused by the delivery of the saline was compared with the artifact caused by the delivery of the insulin. The artifact was quantified as the area under the glucose measurement curve immediately following an insulin or saline delivery. Since the glucose measurement curve has glucose on the y-axis in mmol/L and time in minutes on the x-axis, the units of AUC on this curve would normally be given as mmol/L min. However, it was normalized by the time window over which it was measured, so the final units of AUC remain in mmol/L. The size of the sensor artifact was evaluated for large meal boluses for insulin and saline (same volume delivered as insulin) and compared the size of these artifacts relative to smaller doses that are typical for basal insulin that were given every 5 minutes during the study. For meal boluses, the AUC was measured as the area under the glucose sensing curve from the time that the meal bolus was administered to 30 minutes after the meal bolus. During basal insulin and saline delivery, the AUC was measured as the area under the glucose sensing curve from the time of the basal delivery to 5 minutes after the delivery.

In addition to quantifying the size of the artifact using the AUC after insulin and saline deliveries, the amount of time required for the sensor was quantified to return to within 10% of its baseline value following either a larger meal bolus or a smaller basal delivery. Because the glucose at baseline is typically changing, it is important to account for the trend in glucose when calculating the time it takes to return to within 10% of the baseline. Rather than using the baseline glucose itself, the baseline glucose projected forward in time based on its trend at the time of the delivery. For the continuous interstitial glucose measurement, a 1-point calibration was performed using the closest preceding YSI reference glucose measurement to the time that the delivery occurred, to estimate the sensor sensitivity. Using this estimated sensitivity and the neighboring YSI glucose data points, an expected trend line of the sensor over a window of time was projected. The settling time was calculated, which is the time required for the sensor to return to within 10% of this trend line following a delivery of insulin or saline.

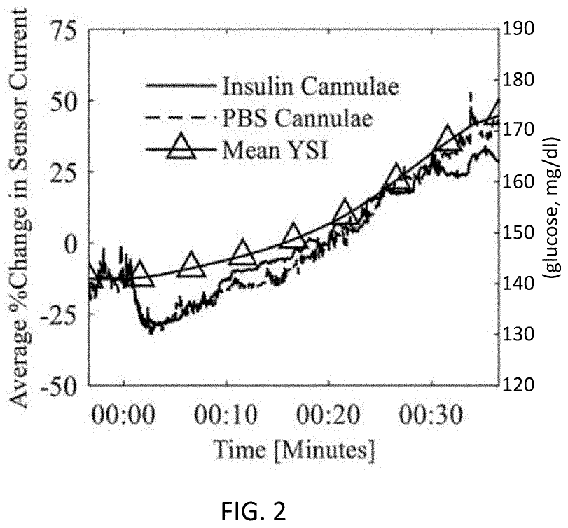

FIG. 2 shows the mean magnitude of the interstitial dilution error after meal boluses of insulin or saline. In this hollow sensing cannula, the indicating electrode was located about 4 mm from the source of insulin delivery. In other configurations of the sensing cannula, there may be more than one indicating electrode. FIG. 2 shows the mean of the reference blood glucose values using a YSI reference glucose machine and the sensor values after insulin and saline. The error (decline below actual glucose value) in sensed glucose occurs very quickly and is maximal at about 2.5 minutes after the bolus. The magnitude of the error averages is variable and averages about −7 to −15 mg/dl. The error begins to decline after the peak, returning upward back to baseline, and is essentially absent at 20 minutes after bolus. At 15 minutes the error is only about −3 to −6 mg/dl. All were given boluses both of saline and aspart insulin via a sensing cannula (see labels: PBS cannulae refers to saline delivery and insulin cannulae refers to aspart insulin delivery). The reference arterialized venous glucose measured by a benchtop Yellow Springs Instruments (YSI) Model 2300 is labeled YSI. The percent change of the sensor signal is on the left Y axis and glucose in mg/dl is on the Y axis. It can be easily seen that the tracings for the insulin and the saline control deliveries were almost identical. However, in both cases, the mean of the sensor signals falls below the actual reference glucose level. This error is referred to as interstitial dilution error.

The benefit of the dilution artifact removal algorithm was analyzed on a study participant using the PDT sensing cannula while the participant dosed insulin through the cannula for repeated meal events across a multi-day study. When not using the LSTM forecasting algorithm to correct the dilution artifact, the mean absolute relative difference (MARD) was 24.2% during the 30-minutes following a meal bolus of insulin. When using the forecasting algorithm to correct for the dilution artifact, the MARD was reduced to 14.2% in the 30-minute post-meal bolus period. The mean relative difference (MRD) also improved from 7.7% to 4.4% in this post-meal period. Across the entire study, including both meal events and non-meal events, the use of the glucose forecasting algorithm was helpful in reducing the MARD by 3-4% overall.

### Summary of Clinical Findings:

The error as measured by AUC after delivery of saline through the sensing cannula was no different from the AUC after delivery of insulin formulation. This finding verified that the most likely cause of the artifact was dilution, that is, dilution of the glucose concentration in the subcutaneous interstitial space. Generally, the direction of the error was negative, that is, below the estimated glucose curve, and thus consistent with dilution. Much less commonly, there was a rise in the estimated glucose and such a finding was consistent with the idea of the fluid bolus (insulin or saline) pushing glucose-containing fluid toward the indicating electrode. In any case, the duration of the artifact seldom exceeded 20-25 minutes. Importantly, the magnitude and duration of the artifact were found to be dependent on the dose of insulin; the larger the bolus dose, the larger the artifact. In contrast to bolus doses given for meals or for hyperglycemia correction, microboluses given for basal control of insulin did not cause a clinically significant artifact. Basal insulin delivery rates for most adults range from 0.3 to 2.5 units per hour, with more typical rates of 0.5-1.5 units per hour.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1.-79. (canceled)

80. A method for estimating a true analyte concentration in a subcutaneous space of a subject, comprising:
(a) delivering a composition into the subcutaneous space;
(b) measuring a first concentration of an analyte in interstitial fluid in the subcutaneous space using a sensor at a first time;
(c) predicting a second concentration of the analyte in the interstitial fluid at the first time using a forecasting model;
(d) combining the first concentration of the analyte and the second concentration of the analyte in a signal processing module to estimate a true analyte concentration; and
(e) repeating (b) to (d) to mitigate a dilution artifact of the measuring responsive to a proximity of the delivered composition to the sensor.

81. The method of claim 80, wherein the composition is delivered within about 15 millimeters (mm) from the sensor.

82. The method of claim 80, wherein the forecasting model is based at least in part on the first concentration of the analyte, previous analyte concentrations measured by the sensor, the delivering of the composition, or any combination thereof.

83. The method of claim 80, wherein (d) further comprises using the signal processing module to determine a weighted sum of the first concentration of the analyte and the second concentration of the analyte.

84. The method of claim 83, wherein the weighted sum is based at least in part on a covariance of the first concentration of the analyte and the second concentration of the analyte.

85. The method of claim 80, wherein the composition is delivered by a tubed pump or a patch pump using a delivery system.

86. The method of claim 85, wherein the delivery system comprises a continuous infusion pump.

87. The method of claim 85, wherein the delivery system comprises an open loop delivery system, a closed loop delivery system, or a hybrid closed loop delivery system.

88. The method of claim 80, wherein (c) further comprises predicting the second concentration of the analyte using the forecasting model in real-time.

89. The method of claim 80, wherein the forecasting model comprises a machine learning model, an ordinary differential equation (ODE)-based model, or a combination thereof.

90. The method of claim 89, wherein the machine learning model comprises a linear regression model, a support vector regression model, a multivariable adaptive regressive spline model, a neural network model, a ridge regression model, a Lasso regression model, or an ElasticNet regression model.

91. The method of claim 90, wherein the neural network model comprises a convolutional neural network, a recurrent neural network, or a combination thereof.

92. The method of claim 91, wherein the recurrent neural network comprises a long-short term memory neural network.

93. The method of claim 89, wherein the ODE-based model comprises an ODE solver to solve a system of ODEs for a time of interest, wherein the system of ODEs comprises kinetics or dynamics of the analyte, the composition, or a combination thereof.

94. The method of claim 89, wherein the ODE-based model comprises a metabolism regulatory model.

95. The method of claim 94, wherein the metabolism regulatory model comprises a glucoregulatory model.

96. The method of claim 89, wherein the machine learning model is trained using training data comprising analyte sensor measurements from a population of subjects with a disease or disorder.

97. The method of claim 80, further comprising signal processing the first concentration of the analyte using a filter to remove noise.

98. The method of claim 97, wherein the filter comprises a low-pass filter, a bandpass filter, a high pass filter, or a combination thereof.

99. The method of claim 80, wherein (d) further comprises the first concentration of the analyte and the second concentration of the analyte using a filter.

100. The method of claim 99, wherein the filter comprises a Kalman filter, extended Kalman filter, or sigma point Kalman filter.

101. The method of claim 99, wherein the first concentration of the analyte is weighted based at least in part on the variance of the measuring.

102. The method of claim 80, wherein the disease or disorder comprises an insulin resistance, Type 1 diabetes mellitus, or Type 2 diabetes mellitus.

103. The method of claim 80, wherein the sensor comprises a continuous amperometric glucose sensor.

104. The method of claim 80, wherein the analyte comprises a carbohydrate.

105. The method of claim 104, wherein the carbohydrate comprises glucose.

106. The method of claim 80, wherein the composition comprises a hormone.

107. The method of claim 106, wherein the hormone comprises insulin, glucagon, pramlintide, or any combination thereof.

108. The method of claim 107, wherein the hormone comprises insulin.

109. The method of claim 80, wherein the composition further comprises a pharmaceutical acceptable excipient comprising phenol, cresol, a salt, a stabilizing agent, or any combination thereof.
